(19) United States
(12) Patent Application Publication
(10) Pub. No.: US 2023/0040463 A1
(43) Pub. Date: Feb. 9, 2023

(54) RISK ASSESSMENT AND INTERVENTION PLATFORM ARCHITECTURE FOR PREDICTING AND REDUCING NEGATIVE OUTCOMES IN CLINICAL TRIALS

(71) Applicant: Y-Prime, LLC, Malvern, PA (US)

(72) Inventors: David James Sjölin, Portland, OR (US); Ian Michael Greenfield, Portland, OR (US)

(21) Appl. No.: 17/817,962

(22) Filed: Aug. 5, 2022

Related U.S. Application Data
(60) Provisional application No. 63/231,217, filed on Aug. 9, 2021.

(51) Int. Cl. G16H 50/30; G16H 50/20; G16H 40/67; G16H 20/00; G16H 70/20; G16H 80/00

(52) U.S. Cl. CPC G16H 50/30; G16H 20/00; G16H 40/67; G16H 50/20; G16H 70/20; G16H 80/00

(57) ABSTRACT

Embodiments of a risk assessment and intervention platform architecture are disclosed, where the risk assessment and intervention platform can predict and reduce negative medical outcomes. Embodiments of the risk assessment and intervention platform may include components with controls or interface elements to receive instructions from clinicians to set parameters for interventions and then to enroll, track, and monitor patient activity through the medical treatment plans. Embodiments of a scoring engine are configured based on a specific medical treatment plan and intervention protocols to initiate intervention as needed.

### LIMITED COPYRIGHT AUTHORIZATION

A portion of the disclosure of this patent document includes material which is subject to copyright protection. The copyright owner has no objection to the facsimile reproduction by anyone of the patent document or the patent disclosure as it appears in the Patent and Trademark Office patent file or records, but otherwise reserves all copyrights whatsoever.

### CROSS-REFERENCE TO RELATED APPLICATION

This application claims the benefit of U.S. Provisional Application No. 63/231,271 titled "Clinical Trial Platform Architecture" filed on Aug. 9, 2021, the entirety of which is hereby incorporated herein by reference for all purposes.

### FIELD OF EMBODIMENTS

This disclosure relates to a risk assessment and intervention platform architecture for reducing negative health outcomes in clinical trials.

### OVERVIEW OF VARIOUS EMBODIMENTS

Various embodiments of systems, methods, and devices are disclosed for providing a platform architecture for reducing negative health outcomes in clinical trials. The systems, methods, and devices of the disclosure each have several innovative aspects, no single one of which is solely responsible for the desirable attributes disclosed herein.

In one embodiment, a system for deployment of one or more risk models for determining intervention risk is disclosed. The system may include: one or more processors; a network communications interface; a memory; and computer code stored in the memory, wherein the computer code, when retrieved from the memory and executed by the one or more processors causes the one or more processors to: access a set of electronic patient medical data associated with a patient and an intervention protocol; access a set of intervention protocol parameters associated with a medical treatment plan in which the patient is actively engaged; automatically generate a linked patient data set based on the set of electronic patient medical data and the set of intervention protocol parameters; apply a deployed machine learning model to generate one or more intervention risk scores associated with a potential harm associated with the patient, wherein the deployed machine learning model is trained using anonymized historical treatment data, wherein the anonymized historical treatment data includes one or more of: historical patient medical data, historical treatment results data; historical patient engagement data; historical clinical data; and for the one or more intervention risk scores that meet a predetermined threshold indicated in the set of intervention protocol parameters, automatically assess the one or more intervention risk scores to select and initiate an intervention action that reduces the potential harm associated with the patient.

In some embodiments, the system may further include computer code stored in the memory, wherein the computer code, when retrieved from the memory and executed by the one or more processors causes the one or more processors to store computer-executable instructions that: initiate an electronic service that actuates a sensor system to collect medical data associated with the patient. In some embodiments, the system may be implemented wherein the collected medical data includes one more of: blood pressure readings, heart rate, glucose level, or oxygen saturation. In some embodiments, the system may be implemented wherein the set of electronic patient medical data associated with the patient includes one or more of: biometric data collected from the patient, patient response data provided by the patient, periodic reporting data indicating an intake of a medication by the patient as compared to a predetermined dosage. In some embodiments, the system may be implemented wherein the deployed machine learning model employs at least one of: a neural network, an adversarial classifier, a k-nearest neighbor algorithm, linear regression, Support Vector Machines, or a Naive Bayes classifier. In some embodiments, the system may further include computer code stored in the memory, wherein the computer code, when retrieved from the memory and executed by the one or more processors causes the one or more processors to store computer-executable instructions that: initiate an electronic service that actuates a notification system to send a secured alert in real-time to a medical clinician indicating that assistance is needed for the patient. In some embodiments, the system may further include computer code stored in the memory, wherein the computer code, when retrieved from the memory and executed by the one or more processors causes the one or more processors to store computer-executable instructions that: initiate an electronic service that actuates a notification system to send a secured alert in real-time to a medical system indicating that the patient is at risk for disengaging with required protocols associated with the medical treatment plan.

In another embodiment, a computer-implemented method of deploying one or more risk models for determining intervention risk is disclosed. The computer-implemented method may include, as implemented by one or more computing devices configured with specific executable instructions to at least: access a set of electronic patient medical data associated with a patient and an intervention protocol; access a set of intervention protocol parameters associated with a medical treatment plan in which the patient is actively engaged; automatically generate a linked patient data set based on the set of electronic patient medical data and the set of intervention protocol parameters; apply a deployed machine learning model to generate one or more intervention risk scores associated with a potential harm associated with the patient, wherein the deployed machine learning model is trained using anonymized historical treatment data, wherein the anonymized historical treatment data includes one or more of: historical patient medical data, historical treatment results data; historical patient engagement data; historical clinical data; and for the one or more intervention risk scores that meet a predetermined threshold indicated in the set of intervention protocol parameters, automatically assess the one or more intervention risk scores to select and initiate an intervention action that reduces the potential harm associated with the patient.

In some embodiments, the computer-implemented method may further include specific executable instructions that: initiate an electronic service that actuates a sensor system to collect medical data associated with the patient. In some embodiments, the computer-implemented method may be implemented wherein the collected medical data includes one more of: blood pressure readings, heart rate, glucose level, or oxygen saturation. In some embodiments, the computer-implemented method may be implemented wherein the set of electronic patient medical data associated with the patient includes one or more of: biometric data collected from the patient, patient response data provided by the patient, periodic reporting data indicating an intake of a medication by the patient as compared to a predetermined dosage. In some embodiments, the computer-implemented method may be implemented wherein the deployed machine learning model employs at least one of: a neural network, an adversarial classifier, a k-nearest neighbor algorithm, linear regression, Support Vector Machines, or a Naive Bayes classifier. In some embodiments, the computer-implemented method further includes specific executable instructions that: initiate an electronic service that actuates a notification system to send a secured alert in real-time to a medical clinician indicating that assistance is needed for the patient. In some embodiments, the computer-implemented method further includes specific executable instructions that: initiate an electronic service that actuates a notification system to send a secured alert in real-time to a medical system indicating that the patient is at risk for disengaging with required protocols associated with the medical treatment plan.

In a further embodiment, a non-transitory computer storage medium storing computer-executable instructions is disclosed that, when executed by one or more processors, cause the one or more processors to at least: access a set of electronic patient medical data associated with a patient and a n intervention protocol; access a set of intervention protocol parameters associated with a medical treatment plan in which the patient is actively engaged; automatically generate a linked patient data set based on the set of electronic patient medical data and the set of intervention protocol parameters; apply a deployed machine learning model to generate one or more intervention risk scores associated with a potential harm associated with the patient, wherein the deployed machine learning model is trained using anonymized historical treatment data, wherein the anonymized historical treatment data includes one or more of: historical patient medical data, historical treatment results data; historical patient engagement data; historical clinical data; and for the one or more intervention risk scores that meet a predetermined threshold indicated in the set of intervention protocol parameters, automatically assess the one or more intervention risk scores to select and initiate an intervention action that reduces the potential harm associated with the patient.

In some embodiments, the non-transitory computer storage medium further stores computer-executable instructions that: initiate an electronic service that actuates a sensor system to collect medical data associated with the patient. In some embodiments, the non-transitory computer storage medium includes instructions that may be implemented such that the collected medical data includes one more of: blood pressure readings, heart rate, glucose level, or oxygen saturation. In some embodiments, the non-transitory computer storage medium includes instructions that may be implemented such that the set of electronic patient medical data associated with the patient includes one or more of: biometric data collected from the patient, patient response data provided by the patient, periodic reporting data indicating an intake of a medication by the patient as compared to a predetermined dosage. In some embodiments, the non-transitory computer storage medium includes instructions that may be implemented such that the deployed machine learning model employs at least one of: a neural network, an adversarial classifier, a k-nearest neighbor algorithm, linear regression, Support Vector Machines, or a Naive Bayes classifier. In some embodiments, the non-transitory computer storage medium further stores computer-executable instructions that: initiate an electronic service that actuates a notification system to send a secured alert in real-time to a medical clinician indicating that assistance is needed for the patient.

### BRIEF DESCRIPTION OF THE DRAWINGS

The Pat. or application file contains at least one drawing executed in color. Copies of this Pat. or Pat. application publication with color drawings will be provided by the Office upon request and payment of the necessary fee.

The foregoing aspects and many of the attendant advantages of this disclosure will become more readily appreciated as the same become better understood by reference to the following detailed description, when taken in conjunction with the accompanying drawings. The accompanying drawings, which are incorporated in, and constitute a part of, this specification, illustrate embodiments of the disclosure. Throughout the drawings, reference numbers are reused to indicate correspondence between referenced elements. The drawings are provided to illustrate embodiments of the subject matter described herein and not to limit the scope thereof. Specific embodiments will be described with reference to the following drawings.

FIG. 1 illustrates a system diagram showing an example embodiment of a clinical trial risk platform.

FIG. 2 illustrates a system diagram showing an example embodiment of an authentication/permissioning system.

FIG. 3 illustrates a system diagram showing an example embodiment of a protocol system.

FIG. 4 illustrates a system diagram showing an example embodiment of a medical data system.

FIG. 5 illustrates a system diagram showing an example embodiment of a risk assessment/intervention system.

FIG. 6 illustrates a system diagram showing an example embodiment of an engagement system.

FIG. 7 illustrates a system diagram showing example embodiments of a machine learning model development system and a machine learning model deployment system.

FIG. 8 illustrates an embodiment of a flow diagram of an invitation authentication process.

FIG. 9 illustrates an embodiment of a flow diagram of an enrollment process.

FIG. 10 illustrates an embodiment of a flow diagram of an intervention process.

FIG. 11 illustrates an embodiment of a flow diagram of an intervention ownership process.

FIGS. 12A, 12B, and 12C illustrate an embodiment of a flow diagram for an engagement process.

FIG. 13 illustrates an embodiment of a flow diagram of a model development process.

FIG. 14A illustrates an embodiment of a user interface for a patient control panel.

FIG. 14B illustrates an embodiment of a user interface for protocol dashboard.

FIGS. 15A, 15B, 15C, 15D, 15E, and 15F illustrates embodiments of user interfaces for clinical trial participant enrollment.

FIGS. 16A, 16B, 16C, 16D, 16E, 16F, and 16G illustrates embodiments of user interfaces for clinical trial participant tracking.

FIG. 17 illustrates an embodiment of a computing system.

### DETAILED DESCRIPTION OF VARIOUS EMBODIMENTS

Embodiments of the disclosure will now be described with reference to the accompanying figures. The terminology used in the description presented herein is not intended to be interpreted in any limited or restrictive manner, simply because it is being utilized in conjunction with a detailed description of embodiments of the disclosure. Furthermore, embodiments of the disclosure may include several novel features, no single one of which is solely responsible for its desirable attributes, or which is essential to practicing the embodiments of the disclosure herein described. For purposes of this disclosure, certain aspects, advantages, and novel features of various embodiments are described herein. It is to be understood that not necessarily all such advantages may be achieved in accordance with any particular embodiment. Thus, for example, those skilled in the art will recognize that one embodiment may be carried out in a manner that achieves one advantage or group of advantages as taught herein without necessarily achieving other advantages as may be taught or suggested herein.

The medical field relies heavily on the use of clinical trials to study patients/participants and how they respond to various treatment plans, protocols, or interventions. These trials are critical to preventing, diagnosing, and treating diseases. Unfortunately, high dropout rates, limited reporting, and underrepresentation are normal in the clinical trial space and some trials involve risk conditions that may cause potential harm or negative outcomes to participants. Some sources have reported that nearly a third of trial participants drop out and that almost half become non-adherent. In addition, it has been found that there are often significant barriers that impact rural residents and people of color. Existing clinical trials are administered with outdated and inflexible procedures, sometimes relying on simple spreadsheets or even paper tracking.

The disclosure provides embodiments of a secured risk assessment and intervention platform architecture for implementing and managing clinical trials which may predict and/or reduce negative outcomes in clinical trials.

The clinical trial risk platform 100 may include components with controls or interface elements to receive instructions from clinicians to set parameters for clinical trials and then to enroll, track, and monitor participant activity through the trials. A scoring engine can be configured based on a specific clinical trial to provide automated alerts to participant and/or clinician devices allowing for engagement and intervention if needed. The clinical trial risk platform 100 may include multiple network interfaces or application programming interfaces to enable and control secured electronic communications with third-party systems, such as, for example, data warehouses, electronic health record systems, identity service management platforms, service provider systems, government agency systems, guardian devices, insurance provider systems, and billing systems. In some embodiments, automated participant tools may also be provided for engaging and motivating participants while at the same time ensuring that the participant's actions are compliant with the parameters of their respective trial(s).

The clinical trial risk platform 100 may also include network interfaces or application programming interfaces to securely communicate with participants, such as, for example, smartphones, smart watches, wearables, Internet-of-things devices, laptops, tablets, smart television systems, automobile systems, fitness equipment devices, glucose monitors, sleep tracking devices, and so forth. In addition, the clinical trial risk platform 100 may include analytic systems that generate or implement machine learning models, such as, for example, to predict behaviors and provide recommended potential interventions or updates to clinical trial parameters. Various embodiments of the secured clinical trial risk platform 100 may boost engagement of participants in the clinical trials, reduce the number of dropouts, and/or increase the accuracy of the clinical trial data.

### Clinical Trial Risk Platform Architecture

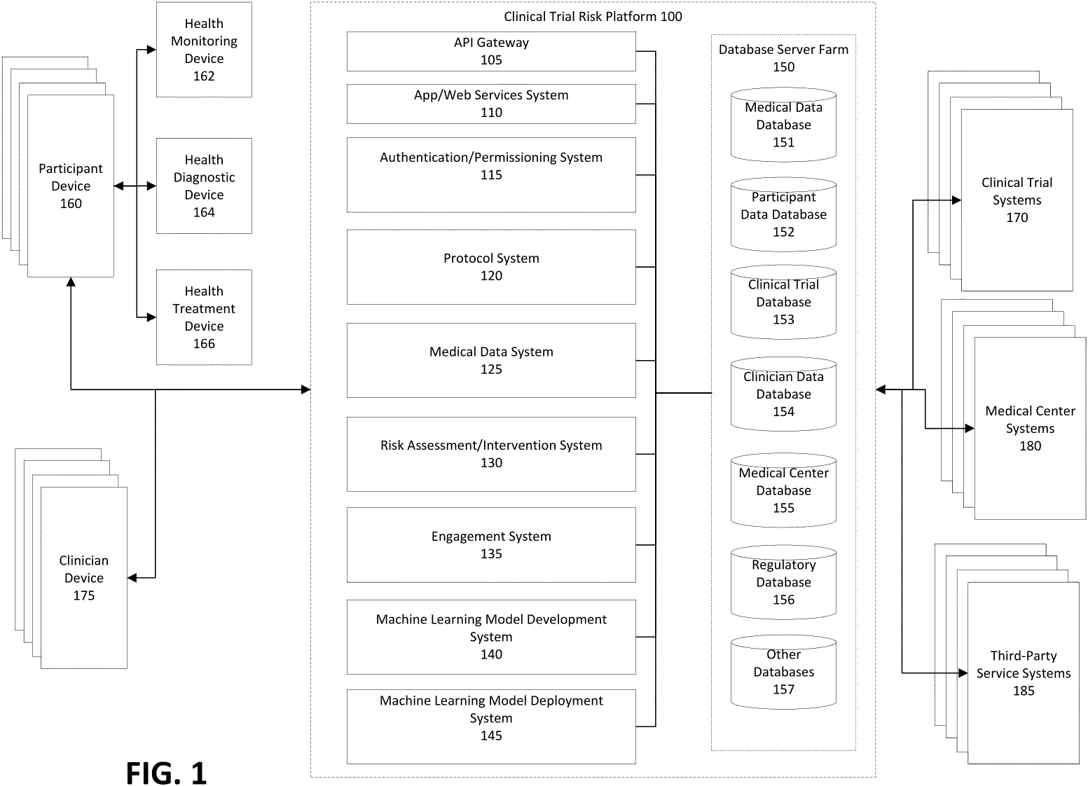

FIG. 1 is a system diagram illustrating an example embodiment of a clinical trial risk platform 100 for implementing and managing clinical trials. In some embodiments, the clinical trial risk platform may predict potential risk as to the participant, determine interventions to reduce or eliminate risk, execute interventions to reduce or eliminate risk, or reduce negative outcomes in clinical trials. The clinical trial risk platform 100 shown in FIG. 1 can electronically communicate with participant devices 160, clinical trial systems 170, clinician devices 175, medical center systems 180, and other third-party service systems 185. Such communication may be over one or more networks (not shown) and may be via communication portals such as application programming interfaces (APIs), electronic portals, file transfer protocol systems, real-time push and pull services, batch delivery, extraction, encryption/decryption services, and so forth. The systems and subsystems of the clinical trial risk platform 100 may also communicate via an internal bus or other technology that transfers data between systems. Moreover, the clinical trial risk platform 100 may make available various data package, messages, or data, such as, for example, via a push interface, a pull interface, a publish/subscribe interface, a blast notification protocol, and so forth.

### Participant Devices

The participant devices 160 include various systems used by potential or actual participants of the clinical trials. The participant devices 160 may include, without limitation, a smartphone, a personal computer, a laptop, a tablet computer, an e-reader device, a car system, a home device system, a smartwatch, a wearable, an Internet-of-things device, a smart television, a fitness device, a medical monitoring or tracking device, a gaming device, or another device capable of connecting to the network to allow communication with the clinical trial risk platform 100. In some embodiments, the participant devices 160 communicate with the clinical trial risk platform 100 via a downloadable application (app), such as, for example, a clinical trial app installed on one of the participant devices 160 or via a website portal accessible via a web browser installed on one of the participant devices 160 that can access the clinical trial risk platform 100.

It is recognized that some participants may want or need to utilize the assistance of others to participate in a clinical trial, such as, for example, minors or participants who are physically or mentally impaired. The clinical trial risk platform 100 may communicate with devices associated with such caretakers and those devices are included in the participant devices 160.

In some embodiments the participant devices 160 may be an electronic communication with other devices such as, for example, health monitoring devices 162, health diagnostic devices 164, and/or health treatment devices 166. For example a health monitoring device 162 may include a device that monitors a participant's heart rate or glucose level, a health diagnostic device 164 may include a device that makes diagnostic decisions such as determining that a glucose level is too low or determining that a heart rate is too high, and a health treatment device 166 may include a device that initiates treatment such as providing insulin to a participant or adjusting a pacemaker of a participant. In some embodiments the health monitoring devices 162, the health diagnostic devices 164, and/or the health treatment devices 166 may communicate with the clinical trial risk platform 100, the clinician devices 175, the clinical trial systems 170, the medical center systems 180, and/or the third-party service systems 185 in addition to or instead of the participant devices 160. These devices may be standalone devices that are provided within the medical field, or they may be consumer devices that are used by consumers for other purposes, such as, for example, smart phones, smartwatches, fitness trackers, and so forth. These devices may collect medical data and/or biological samples from the participants.

### Clinical Trial Systems

The clinical trial systems 170 may include various systems used by entities that are conducting various clinical trials. In some embodiments, the clinical trial systems 170 may store data about various protocols for the clinical trials as well as information about preferred participants, invitation parameters, enrollment requirements and/or processes, interventions, as well as engagement options goals and/or rewards. The clinical trial systems 170 may also include information about historical clinical trials as well as information about prior participants. The clinical trial systems 170 may directly communicate with the clinical trial risk platform 100 to exchange data about the clinical trials. In some embodiments, the clinical trial risk platform 100 may run and/or be part of one or more clinical trial systems 170.

### Clinician Devices

The clinician devices 175 may include various systems used by entities that are providing medical services, such as, for example, doctors, dentists, radiologists, cardiologists, blood testing centers, surgeons, anesthesiologists, physical therapists, nurse practitioners, and so forth. The clinician devices 175 may be used to provide data to the participant devices 160, the clinical trial risk platform 100, the clinical trial systems 170, the medical center systems 180, and/or the third-party service systems 185. In some embodiments, the clinician devices 175 may run clinician apps or website portals that provide information to and collect information from clinicians. Moreover, in some embodiments, the clinician devices 175 may receive alerts or other notifications indicating that an intervention may be required or preferred for one or more participants.

### Medical Center Systems

The medical center systems 180 may include various systems used by medical centers that provide medical services, such as, for example, hospitals, clinics, rehabilitation centers, nursing home facilities, and so forth. The medical center systems 180 may be used to provide data to the participant devices 160, the clinical trial risk platform 100, the clinical trial systems 170, the clinician devices 175, and/or the third-party service systems 185. In some embodiments, the medical center systems 180 may run apps or website portals that provide information to and collect information from within the medical center systems 180.

### Third-Party Systems

The third-party service systems 185 may include other systems used by entities to provide data or services to the clinical trial risk platform 100.

### Clinical Trial Risk Platform

The clinical trial risk platform 100 of FIG. 1 includes an API gateway 105, an app/web services system 110, an authentication/permissioning system 115, a protocol system 120, a medical data system 125, a risk assessment/intervention system 130, an engagement system 135, a machine learning model development system 140, a machine learning model deployment system 145, and a database server farm 150.

### API Gateway

The API gateway 105 may include technology that manages requests being sent out or received via APIs. The API gateway 105 may also perform routing services, detect and time timeout periods, initiate debugging, manage traffic, and perform version control. The API gateway 105 may be used with a variety of APIs including, without limitation, RESTful APIs, Open APIs, internal APIs, partner APIs, JSON-RPC APIs, XML-RPC APIs, SOAP APIs, and WebSocket APIs. The APIs and configurations disclosed in U.S. Provisional Application No. 63/231,271 titled "Clinical Trial Platform Architecture" filed on Aug. 9, 2021 (including in all appendices), are hereby incorporated by reference herein for all purposes.

### App/Web Services System

The app/web services system 110 may include automated services allowing for communication among different devices the Internet or other networks. In some embodiments the app/web services system 110 may listen for requests at a particular part on the network to receive various electronic documents such as HTML, JSON, XML, images, text, and so forth and serve such electronic documents to other systems. In some embodiments, the app/web services system 110 may provide an interface to a database server or other system to allow an application to make requests to and receive data from the database server or other system.

### Database Server Farm

The database server farm 150 of FIG. 1 includes a collection of databases. The example database server farm 150 includes a medical data database 151, a participant data database 152, a clinical trial database 153, a clinician data database 154, a medical center database 155, a regulatory database 156, and other databases 157. While the database server farm 150 illustrates a collection of databases, it is recognized that one or more of the databases may be located external to the clinical trial risk platform 100 and or other data sources may be stored within the clinical trial risk platform 100. In addition, one or more of the datasets within the databases of the database server farm 150 may be combined with other databases and or stored in other portions of the clinical trial risk platform 100.

### Authentication/Permissioning System

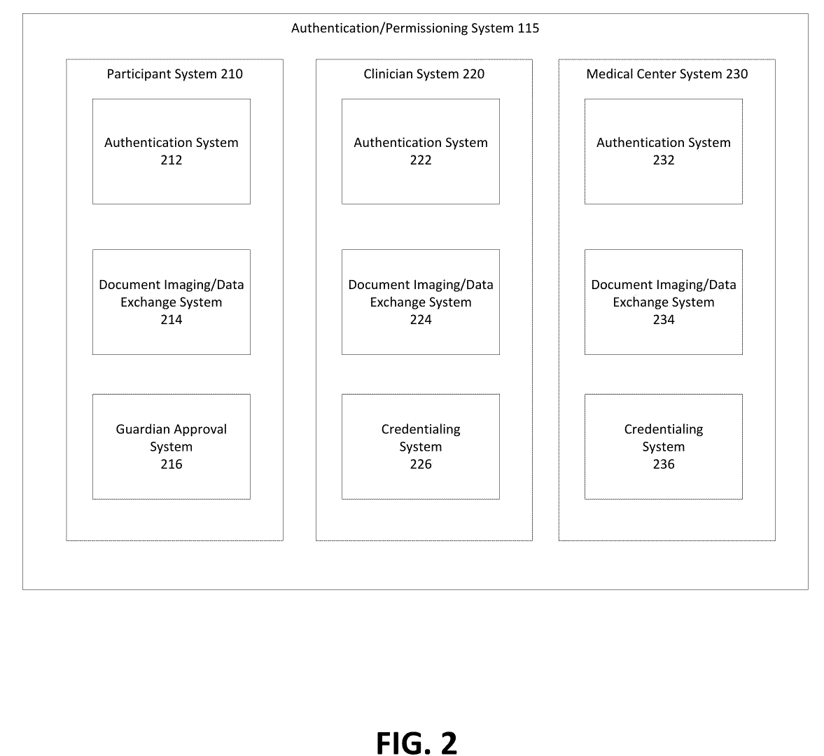

FIG. 2 is a block diagram illustrating an example embodiment of an authentication/permissioning system 115 of the clinical trial risk platform 100 shown in FIG. 1. The authentication/permissioning system 115 that is used to authenticate and provide permissioning for potential or actual participants. In some embodiments, the authentication/permissioning system 115 may also perform authentication and permissioning services for other entities such as, for example, clinical trial administrators, clinicians, medical personnel, and so forth. The authentication/permissioning system 115 of FIG. 2 includes a participant system 210, a clinician system 220, and a medical center system 230.

### Participant System

In some embodiments, the participant system 210 includes an authentication system 212 that provide services for authenticating potential or actual participants. In some embodiments, the participant can personalize the clinical trial app and configure the frequency, style, and content of reminders or interventions. The reminders or interventions may change based on the user's behaviors such as, for example, adherence scores, answers to surveys, demographics, and metadata. In addition, the participant may utilize the clinical trial app to set or to withdraw specific consents or permissions.

The example participant system 210 also includes a document imaging/data exchange system 214 for collecting and analyzing data about the participant. The data may include medical images, such as, for example, x-rays, MRI images, CT Scan images, and so forth. The example participant system 210 also includes a guardian approval system 216 which may provide rules for when guardian approval may be required (for example, for minors or individuals with reduced mental capacity) and the services that are acceptable for obtaining and storing such approval.

### Clinician System

In some embodiments, the clinician system 220 includes an authentication system 222 that provide services for authenticating clinicians and confirming that there is permission from the participant for the clinician to share and receive protected information about the participant and/or the clinical trial. In some embodiments, the clinician can personalize the clinician app and configure the frequency, style, and content of reminders. The reminders may change based on the user's behaviors such as, for example, adherence scores, answers to surveys, demographics, and metadata.

The example clinician system 220 also includes a document imaging/data exchange system 224 for collecting and analyzing data the clinician has about the participant. The data may include medical images, such as, for example, x-rays, MRI images, CT Scan images, and so forth. The example clinician system 220 also includes a credentialling system 226 that may verify the credentials of a clinician, such as, for example, via independent third-party confirmation and/or analyzing scans, photos, or images of the clinician's credential documentation.

### Medical Center System

In some embodiments, the medical center system 230 includes an authentication system 232 that provides services for authenticating representatives of the medical centers and confirming that there is permission from the participant for the medical center to share and receive protected information about the participant and/or the clinical trial.

The example medical center system 230 also includes a document imaging/data exchange system 234 for collecting and analyzing data the medical center has about the participant. The data may include medical images, such as, for example, x-rays, MRI images, CT Scan images, and so forth. The example medical center system 230 also includes a credentialling system 236 that may verify the credentials of representatives of the medical center, such as, for example, via independent third-party confirmation and/or analyzing scans, photos, or images of the representative's credential documentation.

### Protocol System

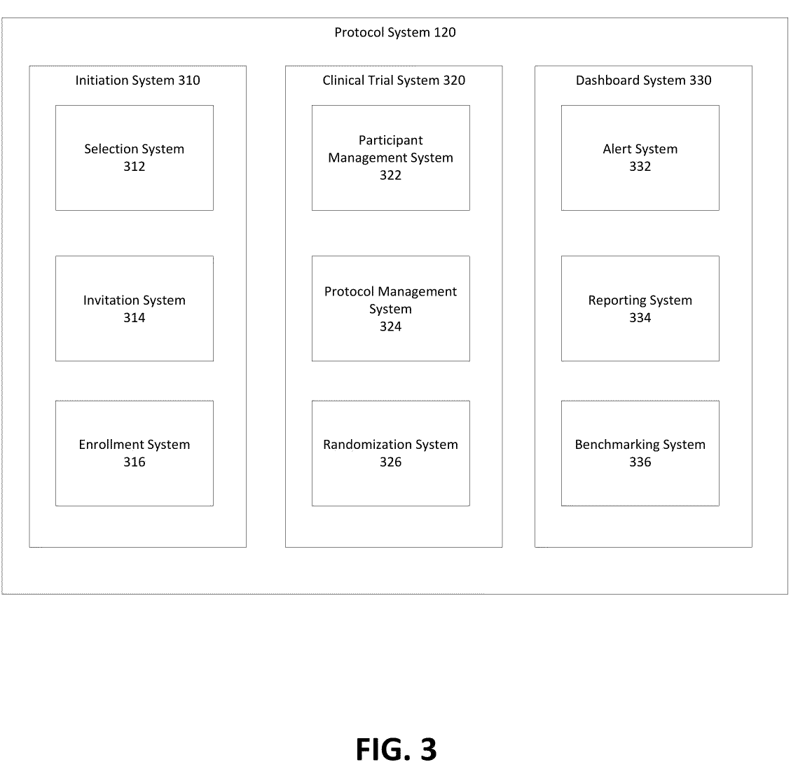

FIG. 3 is a block diagram illustrating an example embodiment of a protocol system 120 of the clinical trial risk platform 100 shown in FIG. 1 that is used to set up, run and manage a clinical trial. The example protocol system 120 includes an initiation system 310, a clinical trial system 320, and a dashboard system 330.

### Initiation System

The initiation system 310 may be used to manage the initiation of a clinical trial with potential participant. In some embodiments, the initiation system 310 includes a selection system 312 that stores parameters and rules associated with the types of potential participants that a clinical trial would like to select and/or invite to participate in the clinical trial. In some embodiments, the initiation system 310 accesses one or more databases that include demographic and other potential participant data.

The initiation system 310 may also include an invitation system 314 that manages the parameters for making invitations available to potential participant and/or tracking the responses (or lack of responses) to such invitations.

The initiation system 310 may further include an enrollment system 316 that manages the parameters and rules associated with the enrollment processes for each clinical trial. The enrollment system 316 may access one or more database that include regulatory requirements that must be met for running certain types of clinical trials as well as reporting requirements for saving and reporting certain data related to certain types of clinical trials.

### Clinical Trial System

The clinical trial system 320 may be used to manage a clinical trial. In some embodiments, the clinical trial system 320 includes a participant management system 322 that manages the activities and status of participants of a clinical trial as well as the preferences of and permissions set by the participants.

The clinical trial system 320 may also include a protocol management system 324 that tracks the particular protocols of the various clinical trials provided by the clinical trial systems and/or their administrators. The protocols may include information and rules about how to run the clinical trial as well as various security parameters to adhere to during the trial. In some embodiments, the clinical trial system 320 includes protocols used to motivate a site or a participant to adhere to standards set by a sponsor or a clinical trial administrator. The clinical trial system 320 may include one or more of the following protocol options:
Contact, follow-up with, or reach out to users;
Schedule users;
Screen users;
Enroll users;
Enter user data; and
Engage in other activities.

The clinical trial system 320 may further include a randomization system 326 that can be run for participants to randomly assign participants to a group of participants that will be receiving certain treatment (or no treatment), often referred to as an "arm" or "control arm" of a clinical trial.

### Dashboard System

The dashboard system 330 may be used to provide information about a clinical trial, such as, for example, real-time updates or alerts about the clinical trial and the participants. In some embodiments, the clinical trial app may include instructions to send data back to the dashboard system 330 or a different system to report the participants progress in the clinical trial (or lack thereof), such as via an API of the data source. The example dashboard system 330 includes an alert system 632, a reporting system 334, and a benchmarking system 336.

The alert system 332 may provide automated real-time alerts as to activities managed or tracked by the protocol system 120. These alerts may include for example positive feedback about participants in the clinical trial, the clinical trial itself, the expected success of the trial, as well as potential negative feedback in similar areas.

The reporting system 334 may provide a variety of customizable reports (whether standalone or presented within features of a user interface) that can be electronically delivered or made available to third-party systems such as, for example, the participants, the clinicians, the clinical trial administrators, and so forth.

The benchmarking system 336 may collect anonymized data about other clinical trials within certain designated segments and/or generated segments such that the clinical trial data can be compared with other clinical trials without exposing any confidential or private information from the other clinical trials.

It is recognized that while the example dashboard system 330 is included as part of the protocol system 120, in other embodiments, the dashboard system 330 may include other information in the clinical trial risk platform 100 and may be located in a different system within the clinical trial risk platform 100.

### Medical Data System

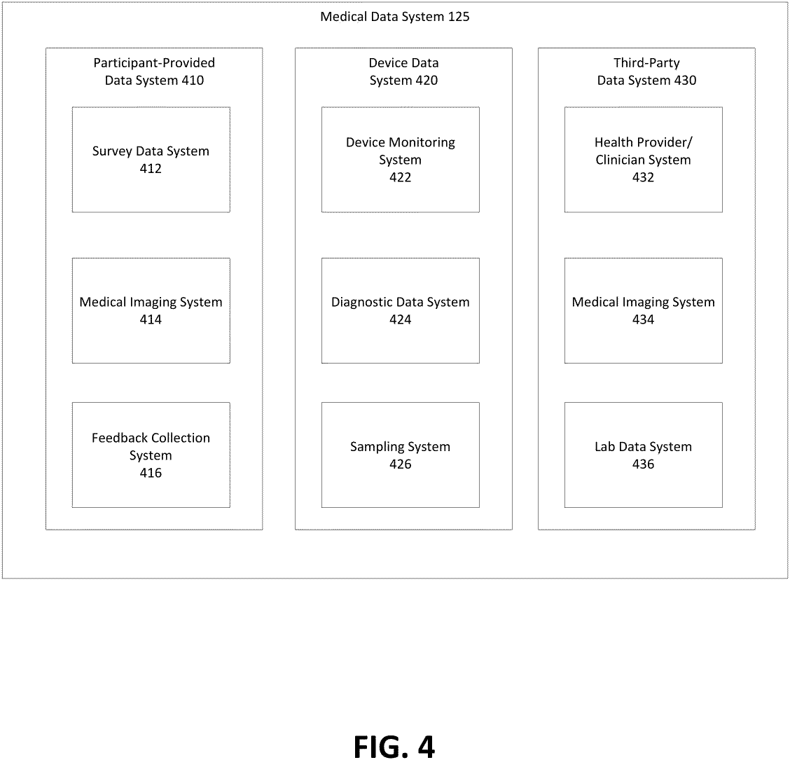

FIG. 4 is a block diagram illustrating an example embodiment of a medical data system 125 of the clinical trial risk platform 100 shown in FIG. 1 that is used to collect, manage, and process medical data of participants in a clinical trial. The example medical data system 125 includes a participant-provided data system 410, a device data system 420, and a third-party data system 430.

### Participant-Provided Data System

The participant-provided data system 410 may be used to manage data collected from or received from the participant and/or the participant devices 160. In some embodiments, the participant-provided data system 410 includes a survey data system 412 that manages data provided by the participant, such as, for example, in response to surveys, in the participant's clinical trial diary, in the enrollment process, and so forth. The participant-provided data system 410 also includes a medical imaging system 414 for collecting and analyzing image data about the participant. The image data may include medical images, such as, for example, x-rays, MRI images, CT Scan images, selfies, personally recorded video, and so forth. The participant-provided data system 410 further includes a feedback collection system 416 that elicits, analyzes, and collects feedback from the participant (as well as the participant's caretaker) about the clinical trial.

### Device Data System

In some embodiments, the device data system 420 can passively pull or actively request biometric or other data from consumer wearable devices, medical devices, other devices, and applications. This data can be encrypted and made available to the clinical trial platform, customer databases, and/or partner databases. This data can be used to indicate health status or intervention efficacy to a researcher or physician, train machine learning models, and/or or other purposes. In some embodiments the device data system 420 generates instructions that initiate or actuate a sensor system to collect medical data associate with the participant. f

The device data system 420 may also manage medical data collected from or received from the participant devices 160, the health monitoring devices 162, the health diagnostic devices 164, and/or the health treatment devices 166. In some embodiments, the device data system 420 includes a device monitoring system 422 that passively pulls or actively requests biometric or other data from consumer wearable devices, medical devices, other devices, and applications. The device data system 420 may also include a diagnostic data system 424 for collecting and analyzing diagnostic data. The device data system 420 may further include a sampling system 426 that sample medical data that is accessible by the medical data system 125.

### Third-Party Data System

The third-party data system 430 may be used to process and manage medical data collected from or received from third parties. In some embodiments, the third-party data system 430 includes a health provider/clinician system 432 that processes and manages medical data provided by the medical center systems 180, the clinician devices 175, and/or other third-party service systems 185 that may have medical data about participants. The third-party data system 430 may access data from health information systems or electronic medical records systems. The third-party data system 430 also includes a medical imaging system 434 for collecting and analyzing image data about the participant received from third-party systems. The image data may include medical images, such as, for example, x-rays, MRI images, CT Scan images, selfies, stored video, and so forth. The third-party data system 430 further includes a lab data system 436 that processes and collects data from various lab systems.

### Risk Assessment/Intervention System

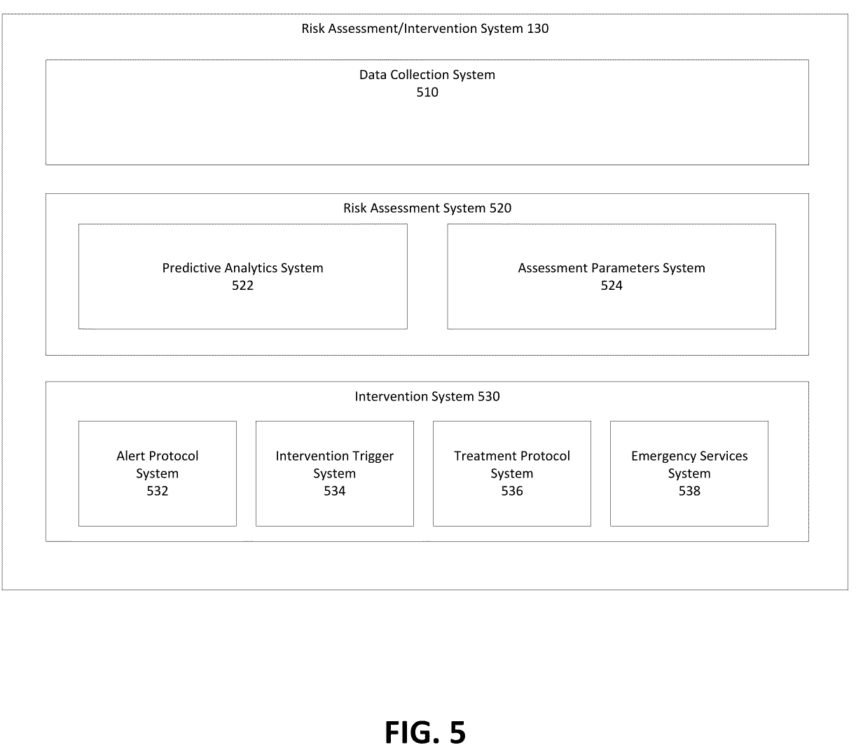

FIG. 5 is a block diagram illustrating an example embodiment of a risk assessment/intervention system 130 of the clinical trial risk platform 100 shown in FIG. 1 that is used to assess potential risk as to participants as well as implement and initiate interventions, if needed. The example risk assessment/intervention system 130 includes a data collection system 510, a risk assessment system 520, and an intervention system 530. The data collection system 510 may process and manage data collected by the clinical trial risk platform 100 that provides information about the participant and/or the related clinical trial.

### Data Collection System

The data collection system 510 may feed data into or make available data to the risk assessment system 520 and/or the intervention system 530. The data collection system 510 may determine that certain information is missing and then generate or initiate requests for the missing information, whether from the clinical trial risk platform 100 or an external system. In some embodiments, the data collection system 510 may track user information including one or more of the following: responses to interventions, including subject and/or user-reported measures according to the study's protocol; demographic data including age, gender, race, comorbidities, and psychographics; activity in the clinical trial app including logging into the clinical trial app, screen views, articles read, and how much time the user spends in the clinical trial app; metadata including time to responses and the time of day when the user engages with the clinical trial app; and biometric data.

### Risk Assessment System

The example risk assessment system 520 includes a predictive analytics system 522 and an assessment parameters system 524. The predictive analytics system 522 may run predictive analytics, such as, for example, deployed machine learning models using received participant data to determine risk levels for a particular participant or a group of participants. The assessment parameters system 524 may include operations that analyze the results of the predictive analytics system 522 alone or in combination with other data about the participant and/or the clinical trial to determine whether there are any potential risks that should be flagged and/or addressed based on the parameters associated with the clinical trial protocol. The predictive analytics system 522 may also determine confidence levels of such risk assessments or other metrics related to those assessments. In some embodiments, the risk assessment system 520 can periodically or continually generate a collection of scores measuring a variety of factors, such as, for example, protocol adherence, retention likelihood, likelihood of adverse event, and/or other risk factors. The scores can be based on provided data or metadata collected including time to respond to intervention, frequency of snoozing or skipping interventions, interaction with other features in the clinical trial app, intervention responses, or other data. Adherence scoring can be broken out or segmented by user, by arm, by site, or by another subgroup and reported in de-identified aggregate to the sponsor and/or clinical manager.

In some embodiments, the risk assessment system 520 can run draft protocols through adherence scoring to predict the likelihood of success and may integrate with the machine learning model development system 140. The risk assessment system 520 can also improve or optimize protocols to improve adherence. Draft protocols for proposed interventions can be provided by study sponsors before the study has begun. The risk assessment system 520 can compare the draft interventions to similar interventions with known adherence data to determine the probability that the draft protocol will or will not experience compliance issues with participants. The risk assessment system 520 can recommend engagement strategies that were effective in improving adherence for the previous, similar interventions.

### Intervention System

The intervention system 530 may initiate interventions, such as, for example:
Medication reminders and dosages;
Surveys;
Appointment scheduling;
Biometric prompts (for example, blood pressure, heart rate, glucose level, oxygen saturation, and so forth); and/or
Other interventional or observational requirements of the trial protocol.

The intervention system 530 may determine that an intervention should be initiated based on parameters of clinical trial protocols, such as, for example, preset rules and predetermined threshold. For example, in some embodiments, the intervention system 530 may make an assessment based on the amount of medication the participant has ingested or taken in comparison to the predetermined dosage the participant should be taking under the clinical trial. The intervention system 530 includes an alert protocol system 532, an intervention trigger system 534, a treatment protocol system 536, and an emergency services system 538. The alert protocol system 532 may access information about the particular protocols that should be applied for a particular clinical trial and/or a group of participants. The intervention trigger system 534 may determine whether and intervention should be triggered, such as, for example, if there is potential or actual harm to a participant or there is risk of disengagement with or from a protocol. The determination may be based on the information from the risk assessment/intervention system 130, such as, for example, the assessment parameters and the alert protocols. If an intervention should be triggered, the intervention trigger system 534 may generate a secure intervention data package, alert, and/or information that can be provided to the appropriate entities. In some embodiments, the intervention trigger system 534 generates instructions that actuate a notification to send a secured alert in real-time. The treatment protocol system 536 may determine specific treatments to initiate or recommend based on assessments made by the risk assessment/intervention system 130. For example, the treatment protocol system 536 may determine that certain actions should be taken, such as, for example, administration of insulin to a diabetic patient or intake of water for a patient that may have symptoms of dehydration. The emergency services system 538 may determine if an emergency event has been detected such that immediate contact should be made to one or more emergency services providers. The emergency service providers may include emergency medical technicians, ambulance services, 911 services, hospital services, and so forth. The emergency services system 538 may also generate and provide notifications to recipients that have been identified as emergency contacts for the corresponding participants.

In some embodiments, the intervention system 530 can expose the intervention to the user from a native protocol management service or proxy intervention information from a remote third-party intervention service such as an Electronic Data Capture (EDC). In some embodiments, protocol and/or user responses to an intervention can be returned to the clinical trial platform 100. The responses may be encrypted, and the intervention system 530 may syndicate these responses to a third-party system, a data warehouse, an EDC, a machine learning model, or any other system.

In some embodiments, the clinical trial app or website generates interfaces that graphically depict data regarding interventions which can include, for example, general reminders or alerts, reminders about medication, surveys, appointment scheduling, biometric readings, and/or diary entries. The reminders can be "snoozable" where the user can close or hide the reminder until it reappears at a later specified time based on the configuration settings. The clinical trial app or website may recognize skipped doses and/or remove reminders after a specified amount of time which is based on the configuration settings. Some interventions may be conditional. For example, some survey interventions may only trigger after the user has taken some number X doses of medication, or after some time Y has passed. Alternatively, for example, a diary entry or survey response may prompt an appointment scheduling intervention or follow-up.

### Engagement System

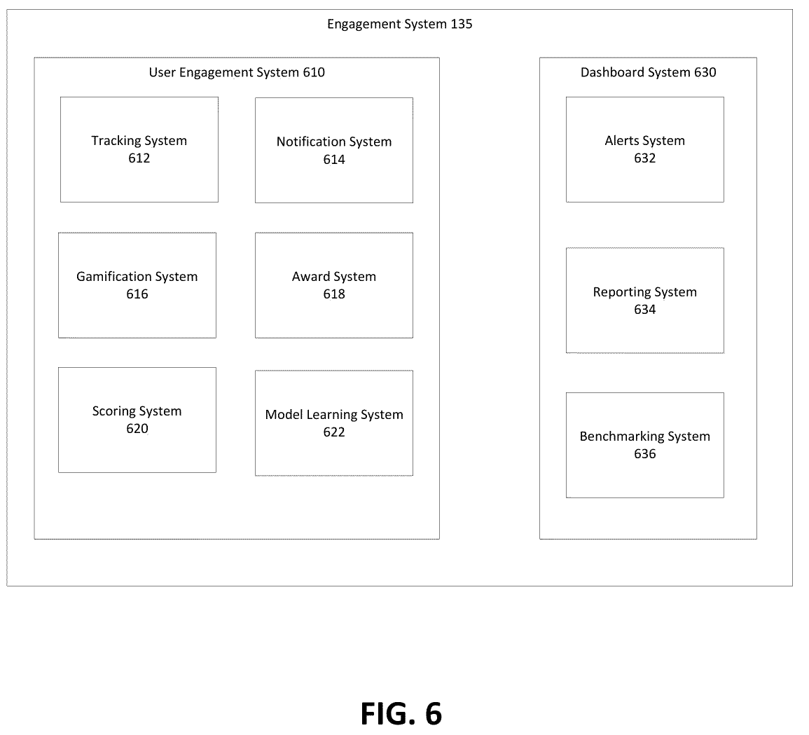

FIG. 6 is a block diagram illustrating an example embodiment of an engagement system 135 of the clinical trial risk platform 100 shown in FIG. 1 that is used to track and improve engagement by users of the clinical trial risk platform 100 including, for example, potential participants, participants, caretakers, research coordinators, study sponsors, clinicians, monitors, and so forth. The example engagement system 135 includes a user engagement system 610 as well as a dashboard system 630.

### User Engagement System

In some embodiments, the user engagement system 610 is configured to communicate with the clinical trial app or website portal to implement features that motivate users with indicators that show a user's progress toward defined achievements; user activities that may trigger achievements; and/or anonymized data for comparison with other users. The achievements may optionally be awarded, for example, in response to logging in to the clinical trial app, the user being in the top A percentile in category B for timeframe C, maintaining a streak of continual protocol adherence, or having the greatest improvement over timeframe C. The user engagement system 610 may also motivate users with a progress indicator toward an achievement. An achievement may trigger an event or award such as a badge, material reward, email, animation, status, or other progress indicator. In some embodiments, the clinical trial app can daisy-chain achievements for more awards. In addition to incremental awards for each of the weeks of perfect adherence a user could receive an award for having had perfect adherence in three of the last four weeks.

The example user engagement system 610 includes a tracking system 612, a notification system 614, a gamification system 616, and award system 618, a scoring system 620, and a model learning system 622.

In some embodiments, the tracking system 612 includes operations that can track user activity and engagement with the clinical trial risk platform 100 as well as the clinical trial app or the website portal. The tracking system 612 may gather data provided by users, metadata, device data, and other data.

In some embodiments, the notification system 614 generates rules that can trigger the generation and execution of notification messages or actions. These notifications may include for example e-mail notifications, text messages, pager alerts, loudspeaker system messages, automated phone calls, flags in dashboards, flashing lights, and so forth.

In some embodiments, the gamification system 616 includes processes that can be used to interface with a user such that the users experience is like a game, a social setting, a social media environment, and online community, and so forth. In some embodiments, the gamification system 616 can apply a rules engine to increase engagement that analyzes user activity and/or triggers specific rewards, notifications, reminders, and/or gamification elements in order to optimize or increase the user's engagement level. The goal criteria rulesets can include points and/or awards such as, for example, one or more of the following:
Buy X Items Y Times (for example via an eCommerce system or component);
Complete User Profile (for example, from a selection of items that define a "complete" profile);
Earn Point Activity X Times (for example, after the user has complete the 100th dosage);
Earn X Out of Y Point Activities (for example, perform some subset of a collection of actions at least once);
Earn X Point Activities in A Row (for example, perform some daily activity X days in a row);
Earn X Point Activity at Y Increment (for example, perform activity X in the 3rd slot of an ordered list of Y activities);
Earn X Points from Point Activity (for example, simply reach some level of points for a given activity);
Earn X Points from Y Point Activities (for example, like above but from a collection of Y activities);
Generic Counter (for example, not tied to an activity - the goal is manually incremented, can be time bound);
Manually Awarded Only (for example, manually awarded by the clinician); and/or
Set Profile Property (for example, a specific profile property is set like Favorite Ice Cream).

In some embodiments, the clinical trial risk platform 100 can include role-based architecture and/or personalization. For example, as users engage with the clinical trial app, the user's actions can trigger the clinical trial app or website portal to assign a role to the user. Roles may be assigned based on a combination of factors, by the goal criteria ruleset, and/or machine learning models. A role assignment can be set to expire which could remove the access the user enjoyed for special features, accelerate scoring, and so forth. As roles build the profile of a user, the clinical trial risk platform 100 can make available new content to the user, creating a personalized experience to best optimize or improve actions from that user. Roles can be assigned via one or more of the following:
Triggers (inside or outside the clinical trial risk platform 100);
Interventions;
Behaviors;
Responsiveness;
Patterns; and/or
Daisy-chained activities.

In some embodiments, roles can be further categorized into "groups" which can be assigned to a user as they progress through the journey. As one example, role groups can include one or more of the following:
Key:
   Role Group
      Role
   Point Level
      Bronze
      Silver
      Gold
      GOAT
   Streaks
      Intervention Logging Streak - 2 days
      Intervention Logging Streak - 1 week
   Milestones
      Clinic Visit - Halfway Done
      10 Daily Diary Logging
      App Log In - Every Week for a Year
      Confirm Final Medication Receipt
   Social Proof
      Article Readers - Top 30%
      Intervention Logging - Top 30% Weekly
      Fast Logging Bonus - Top 30% Weekly
   User Type
      Participant
      Research Coordinator
      Study Sponsor
      Monitor

In some embodiments, the award system 618 tracks and issues awards based on the other systems, such as, for example, the gamification system 616 or the scoring system 620. The awards may be given value within the clinical trial risk platform 100 such as earning access to other areas, getting free or reduced pricing to certain features, or accessing reserved interface elements that can only be accessed if an award is issued. The awards may also include items of value outside of the clinical trial risk platform 100 such as, for example, monetary awards, cryptocurrency awards, gift card awards, and so forth.

In some embodiments, the scoring system 620 includes deployed machine learning models that can generate an engagement score. The clinical trial risk platform 100 can utilize the machine learning models to assign an engagement score to users and then flag users who are most at risk of an engagement issue, such as, for example, an adverse effect, nonadherence, non-engagement, and/or dropout. For example, a low score can trigger the clinical trial risk platform 100 to perform one or more actions, such as initiating adaptive personalization features to re-engage the user, initiating new campaigns to re-engage the user, and/or sending alerts to human coordinators to initiate contact with the user.

In some embodiments, the model learning system 622 feeds data to the clinical trial risk platform 100 that is then ingested by the machine learning model development system 140 to generate new models or to tweak or adjust existing models to make them more accurate.

### Dashboard System

In some embodiments, the dashboard system 630 is configured to provide real-time updates to information from the engagement system 135. In some embodiments, the clinical trial app may include instructions to send data back to the tracking system 612 or a different system to report the user's responses, such as via an API of the data source. The example dashboard system 630 includes an alerts system 632, a reporting system 634, and a benchmarking system 636. The alerts system 632 may provide automated real-time alerts as to activities on the engagement system 135. These alerts may include for example positive feedback about participants in the clinical trial as well as potential negative feedback about participants in the clinical trial. The reporting system 634 may provide a variety of customizable reports (whether standalone or presented within features of a user interface) that can be electronically delivered or made available to third-party systems such as, for example, the participants, the clinicians, the clinical trial administrators, and so forth. The benchmarking system 636 may collect anonymized data about other clinical trials within certain designated segments and/or generated segments such that the clinical trial data can be compared with other clinical trials without exposing any confidential or private information from the other clinical trials. It is recognized that while the example dashboard system 630 is included as part of the engagement system 135, in other embodiments, the dashboard system 630 may include other information in the clinical trial risk platform 100 and may be located in a different system within the clinical trial risk platform 100.

### Machine Learning Model Development & Deployment Systems

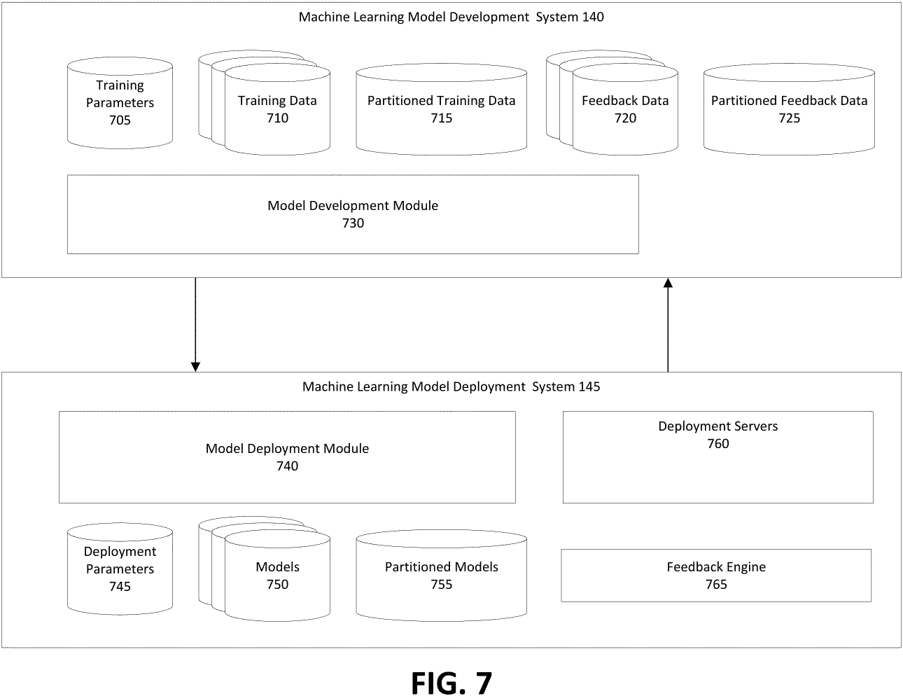

FIG. 7 is a block diagram illustrating example embodiments of a machine learning model development system 140 and a machine learning model deployment system 145 of the clinical trial risk platform 100 shown in FIG. 1 that is used to develop models for the clinical trial risk platform 100 and to deploy and run the models for the clinical trial risk platform 100.

It is recognized that the machine learning model development system 140 may be configured and include components for managing the processing of large data sets that are needed to develop machine learning models, whereas the machine learning model deployment system 145 may be configured differently such that it can timely handle multiple requests for executing models based on the provided data. Thus, in some embodiments, the machine learning model development system 140 may be implemented as a different system from the machine learning model deployment system 145.

### Machine Learning Model Development System

The example machine learning model development system 140 includes training parameters 705 along with training data 710 and partitioned training data 715. In some embodiments, the training data for one clinical trial is stored in a separate database but some training data for multiple clinical trials may be stored in the same database but partitioned physically or logically such that administrators for one clinical trial do not have access to the training data of other clinical trials stored in the same database.

The example machine learning model development system 140 also includes feedback data 720 and partitioned feedback data 725 that is received from or about already deployed models. In some embodiments, the feedback data for one clinical trial is stored in a separate database but some feedback data for multiple clinical trials may be stored in the same database but partitioned physically or logically such that administrators for one clinical trial do not have access to the feedback data of other clinical trials stored in the same database.

The example machine learning model development system 140 also includes a model development module 730 that is used to develop and employ models of different types, such as, for example, a Naive Bayes classifier, a linear regression model, Support Vector Machines (SVM), adversarial classifiers, a k-nearest neighbors algorithm, or a neural network. Other types of machine learning may include, for example, supervised learning, unsupervised learning, reinforcement learning, semi-supervised learning, self-supervised learning, multiinstance learning, inductive learning, deductive inferences, transductive learning, multi-task learning, active learning, online learning, transfer learning, ensemble learning, instance based algorithms, decision tree algorithms, Bayesian algorithms, clustering algorithms, association rule learning algorithms, deep learning algorithms, dimensionality reduction algorithms, and ensemble algorithms.

### Machine Learning Model Deployment System

The example machine learning model deployment system 145 includes a model deployment module 740, deployment servers 760, a feedback engine 765, and deployment parameters 745.

The example model deployment module 740 may be used to manage access to the models that are to be executed as well as track version control. The deployment servers 760 may be used to run the deployed models and may be configured to handle large numbers of execution requests. The feedback engine 765 may collect and manage information and feedback from the executed models and make the information and feedback available to other systems, such as, for example, the machine learning model development system 140.

The example machine learning model deployment system 145 also includes stored models 750 and stored partitioned models 755. In some embodiments, the stored models for one clinical trial are stored in a separate database but stored models for other clinical trials may be stored in the same database but partitioned physically or logically such that administrators for one clinical trial do not have access to the stored models of other clinical trials stored in the same database.

### Other Components

In some embodiments, the clinical trial risk platform 100 can include a companion app or interface (companion component) that is electronically provided to the participant devices 160, such as via an app store. The companion component can include a set of awards based on caretaker behaviors. The interface could be used as a stand-alone app for a partner or in conjunction with a participant clinical trial app.

In some embodiments, the clinical trial risk platform 100 can include a clinician app or interface (clinician component) that is electronically provided to the clinician devices 175, such as via an app store. The clinician component can provide Information about the participants for one or more clinical trials associated with the clinician. The clinician component may generate alerts based on potential risk issues as to the respective clinical trials and/ or the participants. The clinician component may also provide customizable and adjustable user interface components that represent metrics associate with clinical trials and/or the participants.

### Clinical Trial Risk Platform Processes

The clinical trial risk platform 100 may include processes that when executed implement and manage various aspects of clinical trials. The processes may include operations that predict and/or reduce negative outcomes in clinical trials as well as securely communicate with clinicians to set parameters for clinical trials and then enroll, track, and monitor participant activity through the trials. The processes may also include operations that provide automated alerts to clinician devices allowing for engagement and intervention if needed.

It will be appreciated that the embodiments disclosed herein are not limited to the sequence of steps depicted in the flowcharts. In some embodiments, one or more operations of the flowcharts may be combined and performed as a singular operation without calling subprocesses or subdivided. In some embodiments, the inputs and outputs of the operation are passed as values, references, and/or stored in accessible memory locations. In addition, in some embodiments, one or more of the operations may be performed in a different order and/or excluded from the process. In some embodiments, the operations of the processes are executed by one or more components of the clinical trial risk platform 100, but it is recognized that other systems may execute one or more of the operations.

Further, the operations of the flowcharts may be accessed or call and/or data returned via APIs. The APIs and configurations disclosed in U.S. Provisional Application No. 63/231,271 titled "Clinical Trial Platform Architecture" filed on Aug. 9, 2021 (including in all appendices), are hereby incorporated by reference herein for all purposes. Moreover, more than one process may be executed at the same time as other processes or other threads of the same process such that multiple processes may be run at the same time using techniques or combination of techniques, such as, for example, parallel processing, pipelining, or asynchronous I/O.

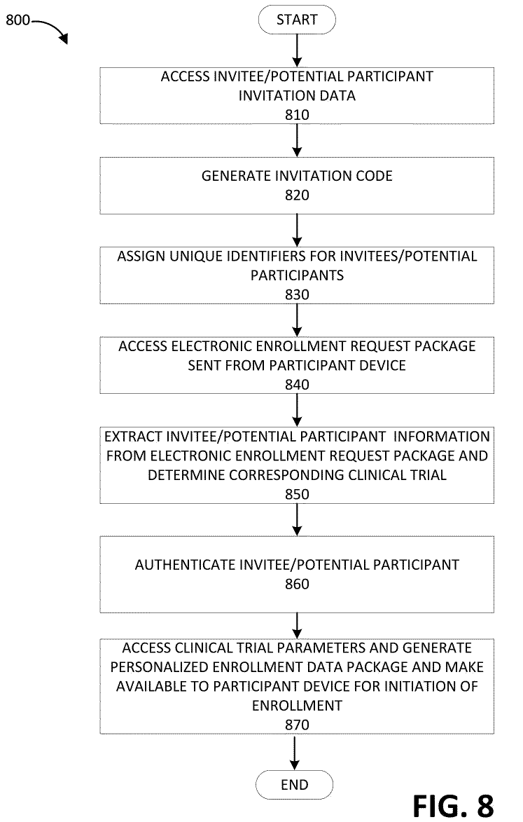

Various embodiments of process of the clinical trial risk platform 100 will now be described with respect to FIGS. 8, 9, 10, 11, 12A, 12B, 12C, and 13. The embodiments include start and end blocks, but it is recognized that other start and end points may be used and that one or more of the processes may be executed as subprocesses of other processes and may not be run as a stand-alone process.

### Invitation Authentication Process

FIG. 8 is a flow diagram illustrating an example embodiment of an invitation authentication process 800. In some embodiments, the invitation authentication process 800 is executed by one or more components of the clinical trial risk platform 100, but it is recognized that one or more operations of the invitation authentication process 800 may be run by other systems.

At block 810, the invitation authentication process 800 accesses participant invitation data. In some embodiments, a potential or prospective trial participant can be recruited from a clinical trial recruitment system and/or site. A system associated with the recruitment system or site (such as, a clinical research site, a contract research organization, a sponsor, and so forth) can securely transmit the participant's information to a system of the clinical trial risk platform 100, such as, for example, the invitation system 314. The recruitment system or site can request an electronic participant invitation from the invitation system 314 which may generate an electronic invitation for electronic delivery to the participant device 160 (which may include a device of the potential participant or potential participant's caretaker as noted above), such as, for example to an app or a website portal accessible via a web browser running on the participant device 160. The participant's information may include and/or determine one or more of the following related to the participant and/or the clinical trial:
Personally Identifiable Information (Pll);
Protected Health Information (PHI);
Eligibility information;
Informed consent documentation;
Electronic signatures;
Onboarding instructions;
Configuration information;
Randomization information;
Trial name;
Provider information;
Contact information; and
Other information as required to streamline and/or facilitate enrollment and onboarding. In some embodiments, the invitation authentication process 800 may be run for a single potential participant for a set of potential participants.

In some embodiments, at blocks 820 and 830, the invitation authentication process 800 may parse the received information to extract and decrypt data items, automatically analyze the data items, and generate an invitation code (such as, for example, a QR code) and/or an invitation link associated with the potential participant as well as a user account and/or a unique identifier associated with the potential participant, which can be then made electronically accessible to the participant. The invitation code or link may be personalized to the potential participant.

In some embodiments, the invitation information can be securely transmitted or made available to the potential participant through the recruitment system or site, such as, for example via an automated message that is pushed to the recruitment system or site via a network component or made available to the participant's account via storage in a data center. The recruitment system or site may then make the invitation information available to the potential participant. In other embodiments, the account information is electronically made available by the clinical trial risk platform 100 to the participant or the participant's caretaker via the participant device 160, such as, for example, via an app or a website portal accessible via a web browser running on the participant device 160.

The app or website portal can display the electronic invitation, or a participant can scan the electronic invitation using a participant device 160 or another device in communication with the participant device 160. The potential participant can instruct the participant device 160 to activate and/or interact with the invitation code or invitation link after receiving it. As one example, if a clinical trial app is already downloaded onto the participant device 160, the QR code or link can automatically launch the clinical trial app. In other scenarios, the QR code or link may include instructions to electronically flag the appropriate app store location to download the clinical trial app.

In some embodiments, after the clinical trial app is launched, it can generate user interface instructions or prompts to guide the potential participant through a customized onboarding process for the specified clinical trial. In some embodiments, the clinical trial app can send a request for enrollment instructions for the invitation code to the clinical trial risk platform 100. At block 840, the invitation authentication process 800 can access an electronic enrollment request package sent by a participant device 160. At block 850, the invitation authentication process 800 may extract and decrypt information from the electronic enrollment request package and determine the clinical trial that is associated with the extracted information. At block 860, the invitation authentication process 800 may authenticate the potential participant to confirm that the person matches the person associated with the corresponding invitation code or link. It is recognized that a variety of authentication operations may be utilized and that some may not require additional input from the potential participant, but that others may require additional input from the potential participant. If the potential participant is unable to be authenticated the invitation authentication process 800 may return an error message and end the process. At block 870, the invitation authentication process 800 accesses the clinical trial parameters associate with the appropriate clinical trial and then generates a personalized enrollment package associated with the potential participant and makes the personalized enrollment package accessible to the potential participant, such as, for example, via the clinical trial app or a website portal via a web browser running on the participant device 160.

### Invitation Authentication Process

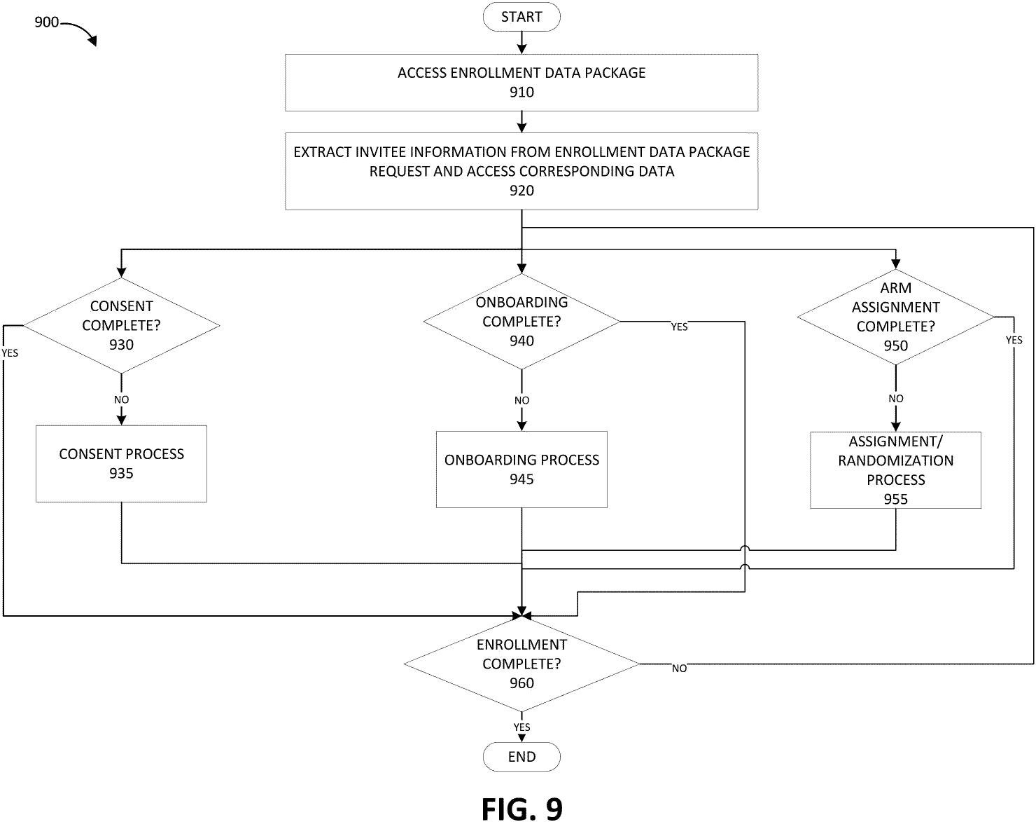

FIG. 9 is a flow diagram illustrating an example embodiment of an enrollment process 900. In some embodiments, the enrollment process 900 is executed by one or more components of the clinical trial risk platform 100, but it is recognized that one or more operations of the enrollment process 900 may be run by other systems.

At block 910, the enrollment process 900 accesses an electronic enrollment data package made accessible by a participant device 160. At block 920, the enrollment process 900 may extract and decrypt information from the electronic enrollment data package and determine the clinical trial that is associated with the extracted information.

In some embodiments, the enrollment process 900 can check to see what information has already been provided by the recruitment system or site, including, for example, any personally identifiable information (PII) of the participant and compare such information with information required by a specific clinical trial. The enrollment process 900 can then guide the participant to provide any remaining, required information, such as via the clinical trial app or the website portal or third-party services. The enrollment process 900 modules may include one or more of the following processes:
Determining eligibility;
Collecting and authenticating informed consent;
Processing credential management (prompting participants to either create a credential or link to a third-party identity system);
Handling randomization into a specific trial arm; and/or
Implementing configuration, including completing an initial questionnaire.

In some embodiments, the enrollment process 900 can determine whether the participant has provided informed consent (block 930), completed instructional onboarding (block 940), and/or if the participant has been assigned a group within a clinical trial (block 95), such as, for example, being randomized into an arm. In some embodiments, an arm includes a group or subgroup of participants in a clinical trial that receives a specific intervention or treatment protocol. In some embodiments, long surveys or onboarding components can be broken down into smaller pieces for potential participants to complete over time.

If the enrollment process 900 does not have an electronic indication of stored informed consent by all required parties, the enrollment process 900 can initiate a consent process to obtain any needed and preferred consents (block 935). The consent process may be run by the clinical trial risk platform 100 and/or may include operations by third parties.

If the enrollment process 900 determines that onboarding has not been completed, the enrollment process 900 can initiate an onboarding process to complete any required or preferred steps for the onboarding (block 945). The onboarding process may be run by the clinical trial risk platform 100 and/or may include operations by third parties.

If the enrollment process 900 determines that arm assignment has not been completed, the enrollment process 900 can initiate an assignment process 955 to complete any required or preferred steps for the onboarding (block 945). In some embodiments, the assignment process 955 includes a randomization process to assign the participant to a specific arm of the clinical trial. The assignment process may be run by the clinical trial risk platform 100 and/or may include operations by third parties.

At block 960, the enrollment process 900 determines whether the informed consent, onboarding, and assignment have been completed. If not, the enrollment process 900 may return to blocks 930, 940, and 950 to determine which portions of the enrollment process are not complete. In some embodiments, the clinical trial risk platform 100,can generate alerts to be provided to or accessible by the participant, the third-party providing consent, and/or the clinicians if the enrollment process has not been completed.

In some embodiments, the enrollment process 900 may generate or update a linked credential and, optionally, two-factor authentication and/or a biometric credential that the participant can use to access the active clinical trial portions of the clinical trial app or website portal after the participant is enrolled.

### Intervention Process

FIG. 10 is a flow diagram illustrating an example embodiment of an intervention process 1000. In some embodiments, the intervention process 1000 is executed by one or more components of the clinical trial risk platform 100, but it is recognized that one or more operations of the intervention process 1000 may be run by other systems.

At block 1010, the intervention process 1000 accesses an intervention trigger data package which includes intervention data about the participant. The intervention process 1000 may extract and decrypt information from the intervention trigger data package and determine participant data and clinical trial data that is associated with the extracted information. At block 1020, the intervention process 1000 accesses intervention parameters associated with the clinical trial to determine whether an intervention action should be initiated, and if so, what intervention action should be initiated. At block 1030, the intervention process 1000 determines whether a local or remote service will be handling the intervention action. If remote, then at block 1035, the intervention process 1000 generates and sends an intervention request package to the appropriate remote system, such as via an API. If local, then at block 1040, the intervention process 1000 executes the intervention. In some embodiments, the intervention may involve operations executing on the clinical trial app or website portal. In some embodiments, the intervention process 1000 may prompt the participant to complete certain actions specified for the flagged intervention. Interventions may include one or more of the following:
Medication reminders and dosages;
Surveys;
Appointment scheduling;
Biometric prompts (for example, blood pressure, heart rate, glucose level, oxygen saturation, and so forth); and/or
Other interventional or observational requirements of the trial protocol.

The participant can provide responses or indicate required actions via the clinical trial app or website portal, which may send an intervention result data package to by accessed by the intervention process 1000.

At block 1050, the intervention process 1000 accesses an intervention result data package which may be from a local system or a remote system. At block 1060, the intervention process 1000 determines if the intervention is complete. If so, at block 1070, the intervention process 1000 logs the intervention. If not, the intervention process 1000 executes intervention escalation procedures 1065 and returns to block 1030.

As noted above, the clinical trial risk platform 100 may include machine learning models. In some embodiments, the machine learning models can process participant responses, clinical trial data, medical data, activities, and/or other data collected by the clinical trial app or accessible by the clinical trial risk platform 100. The machine learning models can be developed and configured to predict the probability a participant is complying with the protocol, the probability the participant may drop out of the clinical trial, the probability the participant will experience an adverse event, as well as other predictions. In some embodiments, the machine learning models are deployed and executed to determine when an intervention may be preferred or required.

### Intervention Ownership Process

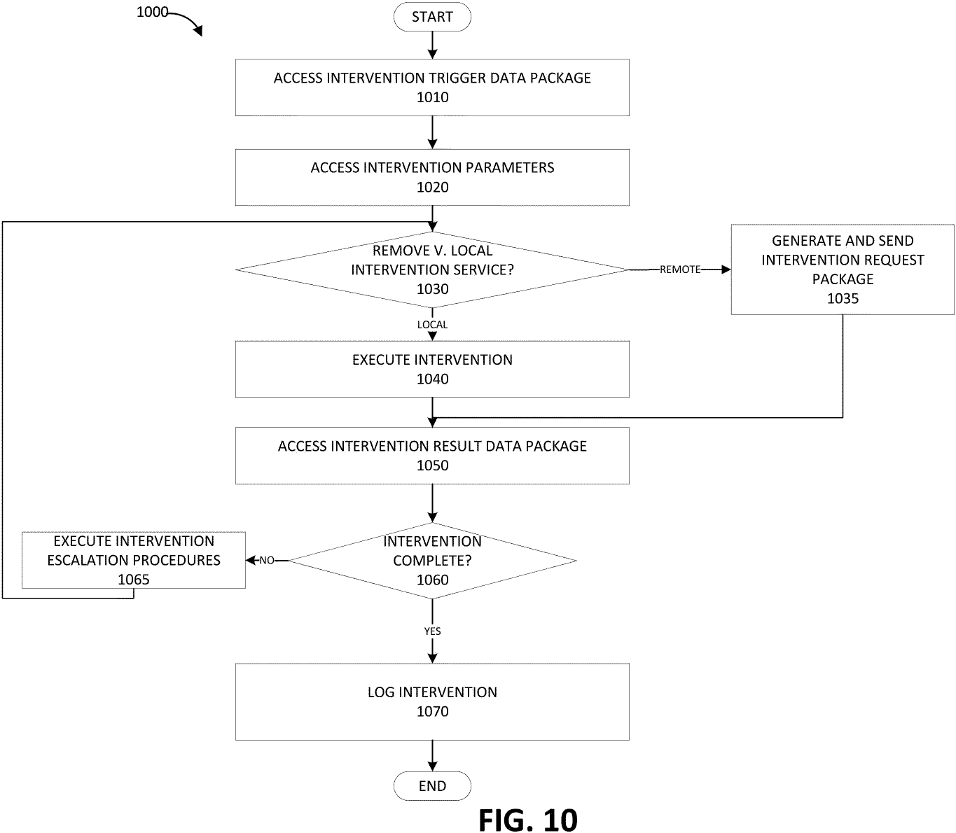

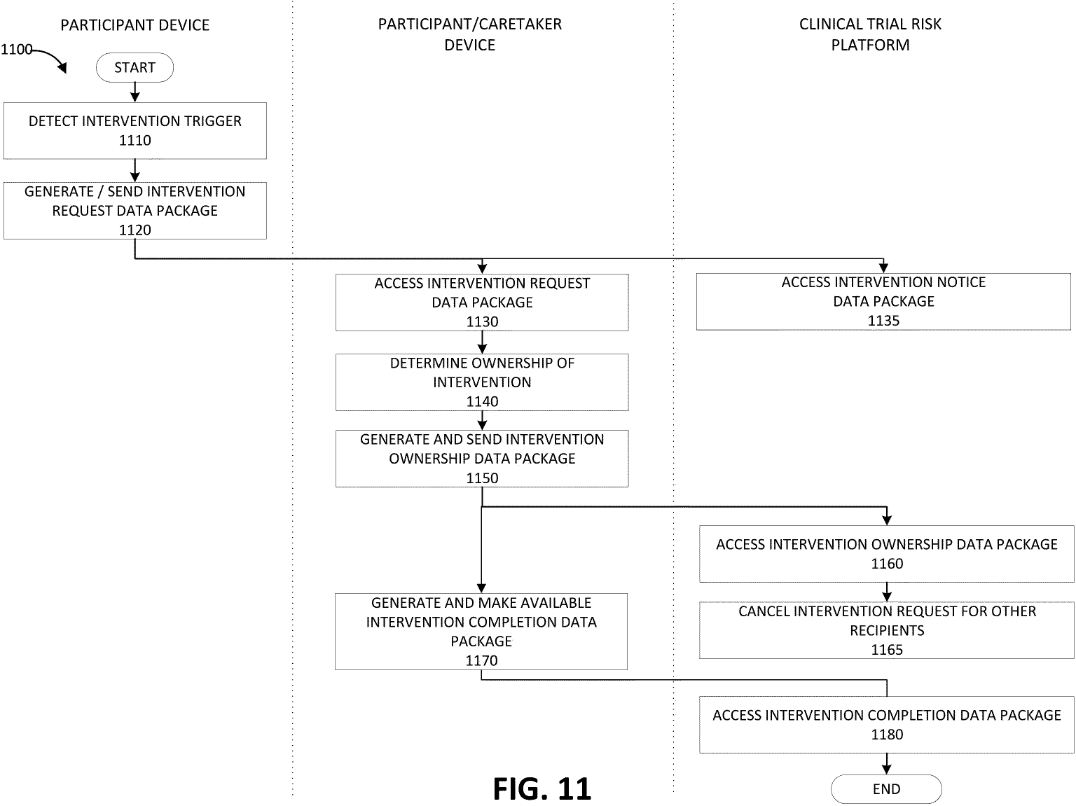

FIG. 11 is a flow diagram illustrating an example embodiment of an intervention ownership process 1100 for determining which entity and related system will be earmarked as responsible for processing and completing the intervention. In some embodiments, the intervention ownership process 1100 is executed by one or more components of the clinical trial risk platform 100, but it is recognized that one or more operations of the intervention ownership process 1100 may be run by other systems.

In block 1110, a participant device 160 may detect an intervention trigger event, and in block 1120, the participant device 160 may generate and send/make available an intervention request data package. The intervention request data package may be accessed by the participant device 160 or another participant device 160 (such as a device of a caretaker) (block 1130), and/or the clinical trial risk platform 100 (block 1135) thereby providing notification of an intervention event. This electronic notification may be provided via the clinical trial app, a companion app, a clinician app, a website portal accessible via a web browser, or another system that provides alerts. At block 1140, one of the participant devices 160 determines ownership of the intervention, and in block 1150, generates and sends an intervention ownership data package. In block 1160, the clinical trial risk platform 100 accesses the intervention ownership data package, and extracts and decrypts the intervention ownership information. The clinical trial risk platform 100 may store data indicating which device, and/or participant or caretaker is the owner of the intervention. At block 1165, the clinical trial risk platform 100 cancels the intervention request for the other recipients, such as by generating and sending a cancellation message. At block 1170, the participant device 160 determines that the intervention has been completed and then generates and makes available an intervention completion data package. At block 1180, the clinical trial risk platform 100 accesses the intervention completion data package and stores information indicating that the intervention is complete. In some embodiments, the clinical trial risk platform 100 may issue an update request to the participant device associated intervention ownership if the clinical trial risk platform 100 has not received an intervention completion data package within a certain timeframe. In addition, other escalation processes may be initiated based on the type of intervention and the severity level. For example, if the intervention is associated with a diabetic condition that may indicate diabetic ketoacidosis, an emergency responder request may be sent to the last known location of the participant device or the participant.

### Engagement Process

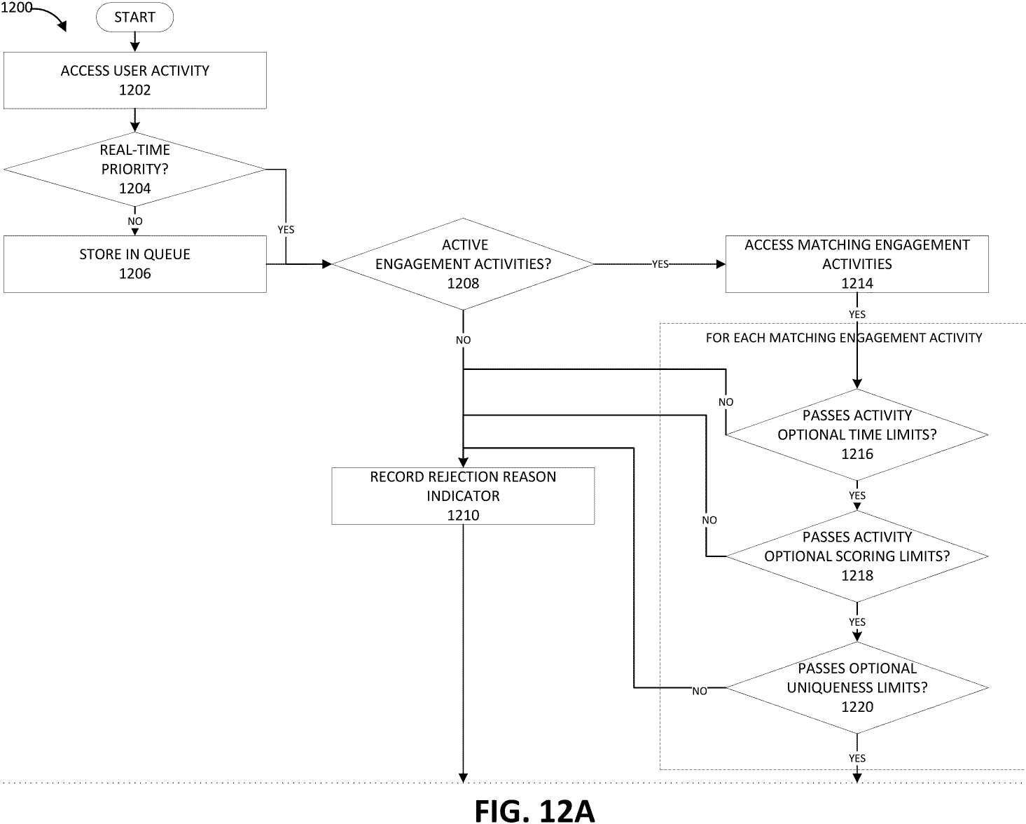

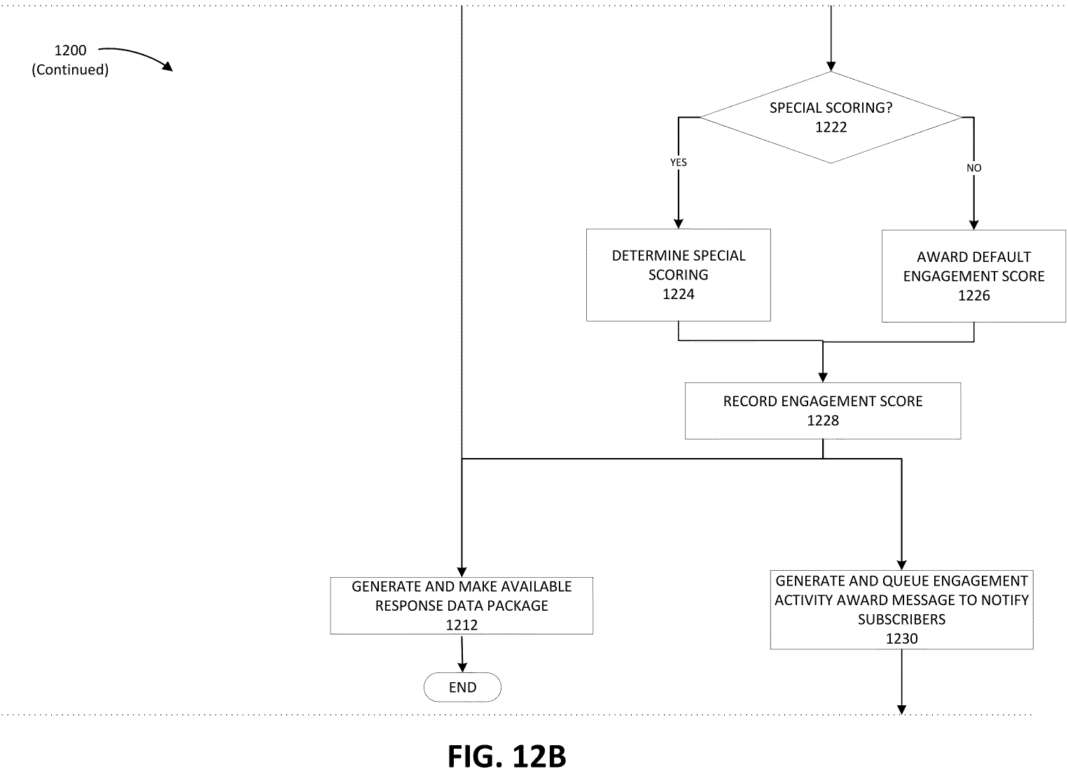

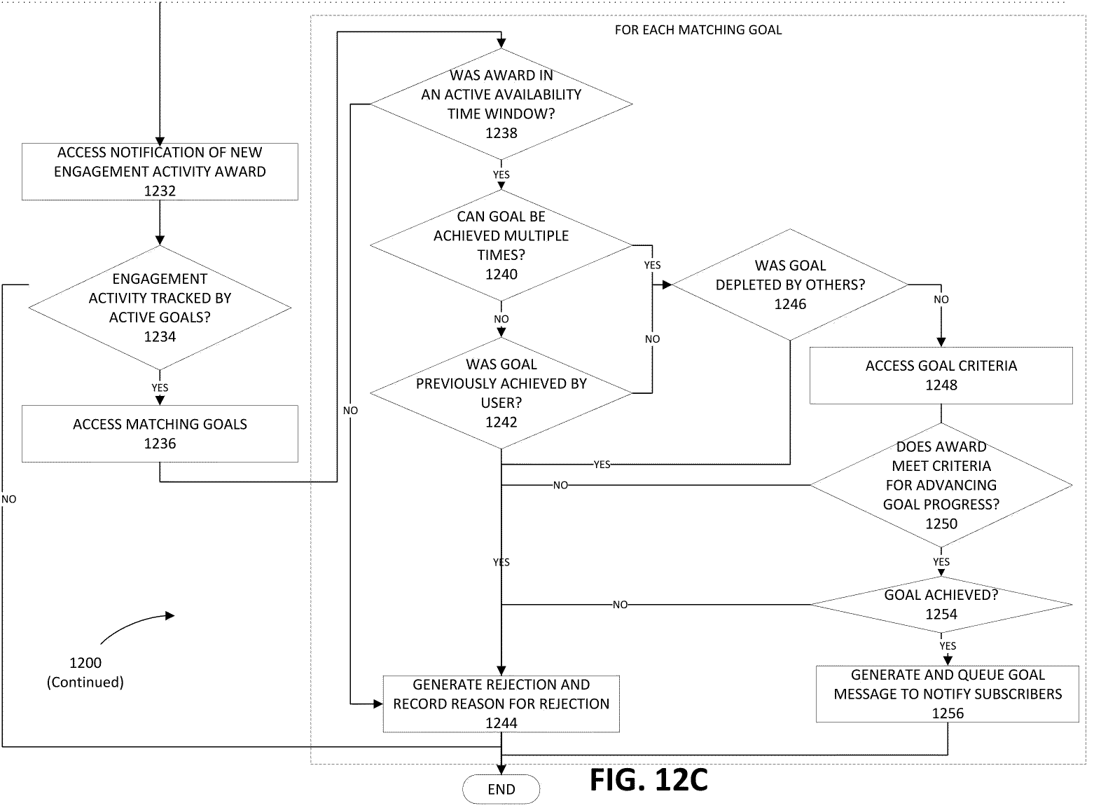

FIGS. 12A, 12B, and 12C are a flow diagram illustrating an example embodiment of an engagement process 1200. In some embodiments, the engagement process 1200 is executed by one or more components of the clinical trial risk platform 100, but it is recognized that one or more operations of the engagement process 1200 may be run by other systems. The engagement process 1200 may be used to track and improve the engagement of a variety of users of the clinical trial risk platform 100 including, for example, potential participants, participants, caretakers, research coordinators, study sponsors, clinicians, monitors, and so forth. The engagement process 1200 may also be used to apply and encourage engagement goals.

In block 1202, the engagement process 1200 accesses user activity data stored in the clinical trial risk platform 100 or received from other systems. The engagement process 1200 can gather user activity reported by a user's clinical trial app, webpage portal, computer, web service, and/or the like. In some embodiments, users can make quick diary entries using voice-to-text. Adverse events can also be reported using voice-to-text. The voice-to-text process may use a third-party service for dictation. The diary entry code terms and sentiment may be validated and may prompt the user to confirm such entries. At block 1204, the engagement process 1200 determines whether there is a real-time priority related to the activity, such as by interfacing with an activity broker. If not, in block 1206, the engagement process 1200 stores the activity in a queue on the clinical trial risk platform 100 for processing at a later time based on other activities already stored in the queue. If yes, in block 1208, the engagement process 1200 determines whether there are any active engagement activities or if user activity matches the active engagement activities. Block 1208 may also be executed in conjunction with activity data that has been stored in the queue that was not a real-time priority but has reached the beginning of the queue. If there are no active engagement activities, in block 1210, the engagement process 1200 records a rejection reason indicator. The engagement process 1200 then generates and makes available a response data package (block 1212). If there are active engagement activities, in block 1214, the engagement process 1200 accesses matching engagement activities, such as, for example, via a validation request. The validation request can include one or more of: a determination whether the activity passes optional time limits, a determination whether the activity passes optional scoring limits, and/or a determination whether the activity passes optional uniqueness limits.

For each matching engagement activity, the engagement process 1200 may determine whether the engagement activity passes optional time limits (block 1216), passes active activity optional scoring limits (block 1218), and passes optional uniqueness limits (block 1220). If any of those checks have not been passed, in block 1210, the engagement process 1200 records a rejection reason indicator. In block 1212, the engagement process 1200 generates and makes available a response data package.

If the checks have been passed, in block 1222, the engagement process 1200 determines whether there is special scoring that should be applied, such as, for example if the user belongs to a group that qualifies for special scoring. If yes, in block 1224, the engagement process 1200 determines the special scoring. If not, in block 1226, the engagement process 1200 awards a default engagement score. In block 1228, the engagement process 1200 records the engagement score. The engagement process 1200 then generates and makes available a response package (block 1212). In block 1230, the engagement process 1200 generates and queues engagement activity award messages to notify subscribers that are subscribed to receive the messages.

In block 1232, the engagement process 1200 accesses a notification of new engagement activity award. In block 1234, the engagement process 1200 determines whether the engagement activity is being tracked by any active goals. If not, then the engagement process 1200 discontinues. If yes, in block 1236, the engagement process 1200 accesses the active goals that match or correspond to the engagement activity.

For each matching goal, in block 1238, the engagement process 1200 determines whether the award was in the active availability time window. If it was not in the active availability time window, in block 1244, the engagement process 1200 generates a rejection and records a reason for the rejection. If it was in the active availability time window, in block 1240, the engagement process 1200 determines whether the goal can be achieved multiple times. If the goal cannot be achieved multiple times, in block 1242, the engagement process 1200 determines whether the goal was previously achieved by the corresponding user. If the goal was previously achieved by the corresponding user, in block 1244, the engagement process 1200 generates a rejection and records reason for the rejection. If the goal can be achieved multiple times or if the goal cannot be achieved multiple times but had not been previously achieved by the corresponding user, in block 1246, the engagement process 1200 determines whether the goal was depleted by others. If the goal was depleted by others, in block 1244, the engagement process 1200 generates a rejection and records a reason for the rejection. If the goal was not depleted by others, in block 1248, the engagement process 1200 accesses goal criteria. In block 1250, the engagement process 1200 determines whether the award meets the criteria for advancing goal progress. If the award does not meet the criteria, in block 1244, the engagement process 1200 generates a rejection and records a reason for the rejection. If the award meets the criteria, in block 1254, the engagement process 1200 determines whether the goal was achieved. If the goal was not achieved, in block 1244, the engagement process 1200 generates a rejection and records a reason for the rejection. If the goal was achieved, in block 1256, the engagement process 1200 generates and queues a goal message to notify subscribers. It is recognized that criteria in FIGS. 12A, 12B, and 12C are example rules and that a variety of rules could be implemented by the clinical trial risk platform 100 which may be different for different trials, different providers, different participant characteristics (such, as for example, age, geographic location, prior history, and so forth) and/or different time periods.

### Model Development Process

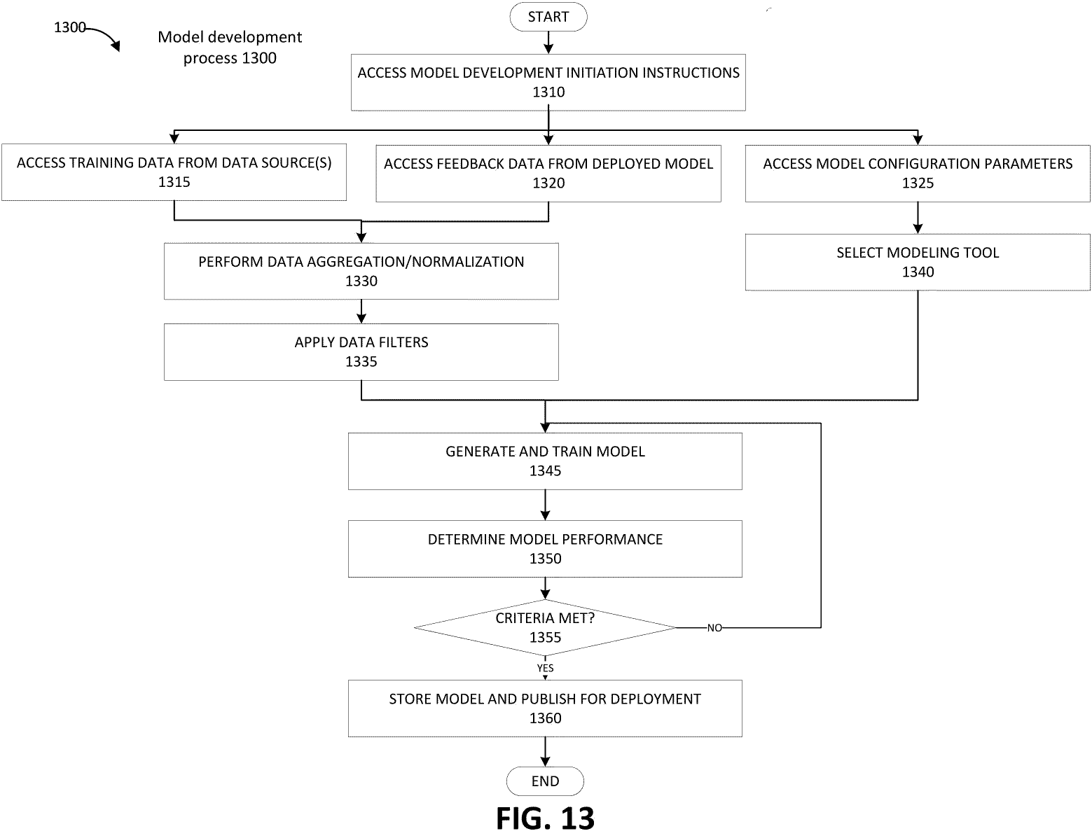

FIG. 13 is a flow diagram illustrating an example embodiment of a model development process 1300. In some embodiments, the model development process 1300 is executed by one or more components of the clinical trial risk platform 100, but it is recognized that one or more operations of the model development process 1300 may be run by other systems. It is recognized that in some embodiments of separate system is used to perform the machine learning model development processes, and after a model has been fully developed such that it meets the final criteria, the model may be exported to a deployment system where the model may be deployed for execution. The system used to develop the model may be configured and include components for managing the processing of large data sets that are needed to develop a model, whereas the deployment system may be configured differently such that it can timely handle multiple requests for run the model based on the provided data.

In block 1310, model development process 1300 accesses model development initiation instructions which may be stored in the clinical trial risk platform 100 or another system. In block 1315, the model development process 1300 accesses training data from one or more data sources which may be locally and/or remotely stored. In block 1320, the model development process 1300 accesses feedback data from a previously deployed model, such as, for example, when the model development process 1300 is being used to further configure and turn an existing model. In block 1325, the model development process 1300 accesses model configuration parameters. The model configuration parameters may include parameters provided by a system administrator and/or by the clinical trial administrator. In block 1330, the model development process 1300 performs data aggregation and normalization of data from the multiple data sources. In block 1335, the model development process 1300 applies data filters to the aggregated and normalized data. In block 1340, the model development process 1300 selects a modeling tool which may include one or more third-party modeling tools and/or custom machine learning modeling tools. It is recognized that different models may be of different types, such as, for example, a Naive Bayes classifier, a linear regression model, Support Vector Machines (SVM), adversarial classifiers, a k-nearest neighbors algorithm, or a neural network. In block 1345, the model development process 1300 generates and trains the model. In block 1350, the model development process 1300 determines the model's performance. In block 1355, the model development process 1300 determines whether the criteria have been met based on the model performance characteristics. If the criteria have not met, the model development process 1300 may regenerate and/or retrain the model in block 1345. If the criteria have been met, in block 1360, the model development process 1300 stores the model and may also publish the model for deployment on the existing system or on a different system.

### Clinical Trial Risk Platform User Interfaces

The clinical trial risk platform 100 may include systems for securely displaying or generating instructions for securely displaying user interface features. The following section discusses embodiments of various user interfaces. While the embodiments provide various features, examples, screen displays, and user interface features, it is recognized that other embodiments may be used. Moreover, the example user interfaces may be displayed on an app (such as, for example, a clinical trial app), on a website portal accessible via a web browser, or a program embedded in a system. Further, the user interfaces and related features disclosed in U.S. Provisional Application No. 63/231,271 titled "Clinical Trial Platform Architecture" filed on Aug. 9, 2021, (including in all appendices) are hereby incorporated by reference herein for all purposes.

### Participant Control Panel

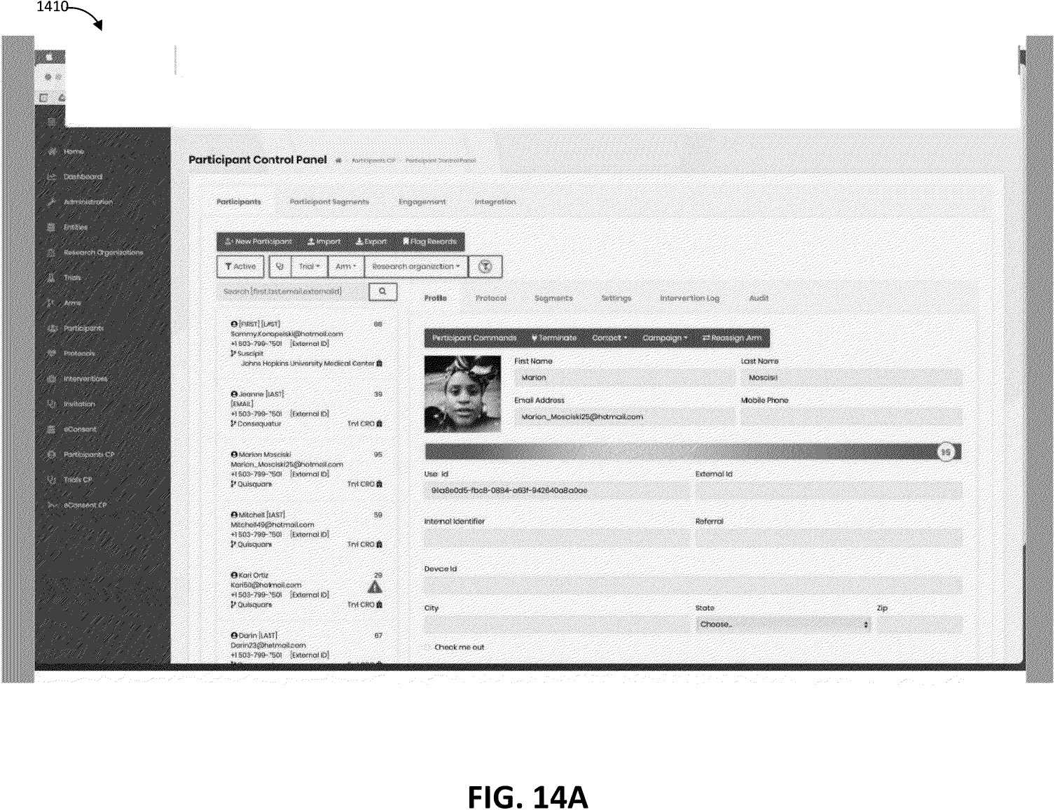

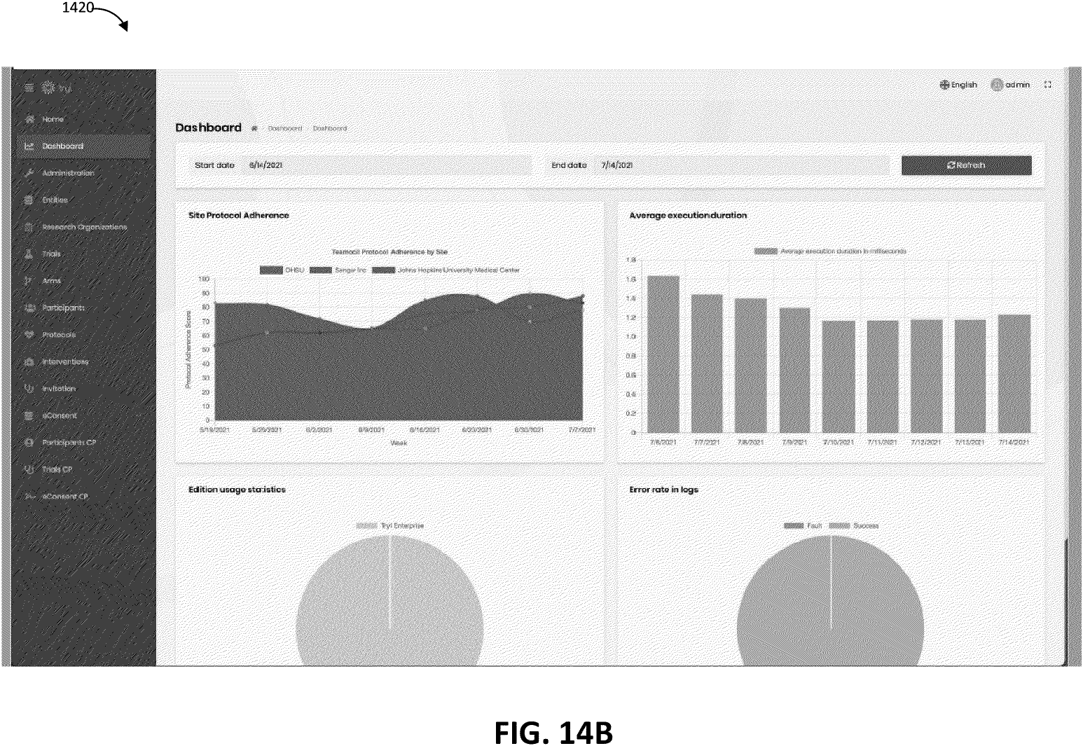

FIG. 14A illustrates an embodiment of an example user interface 1410 for providing a participant control panel. The participant control panel may include information on participants, various protocols, and interventions. The participant control panel may also include permissioning information, consents, electronic consents, as well as other privacy control and permission information associated with the participants. In some embodiments, participant control panel may also include alerts and flags regarding risks and recommended interventions and may be generated and provided in real-time.

FIG. 14B illustrates an embodiment of an example user interface 1420 for providing a dashboard that illustrates information about the protocols including, for example, site protocol adherence, average execution duration, edition usage statistics, and error rate in lags.

### Enrollment

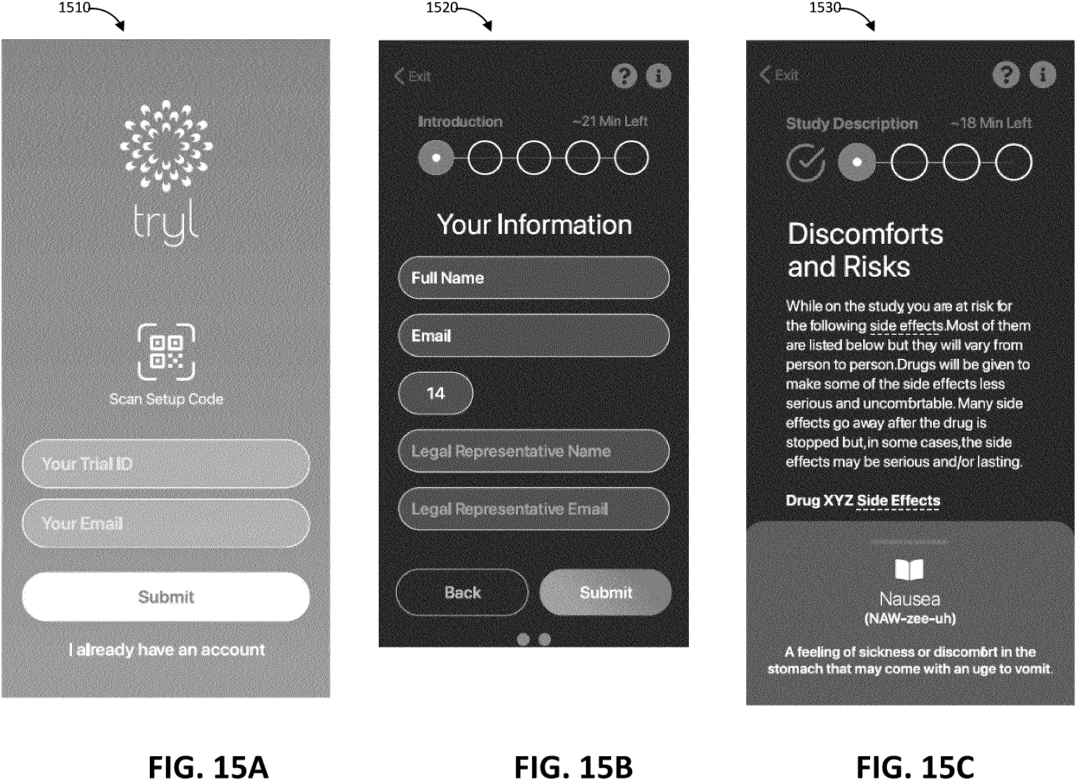

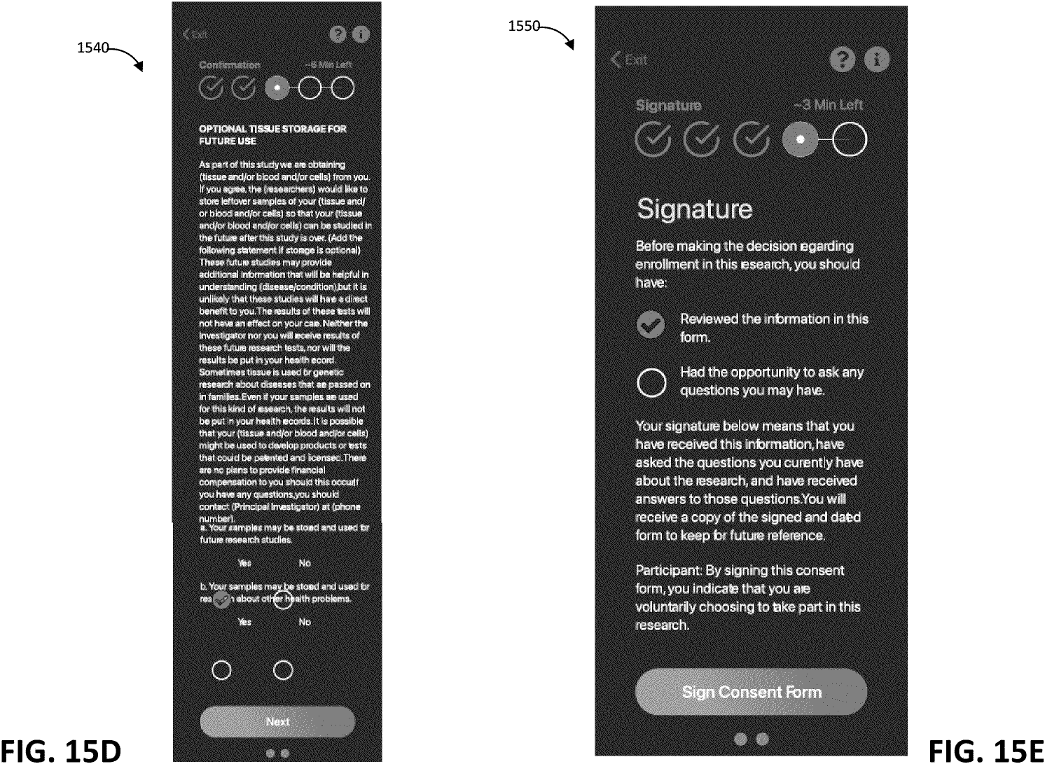

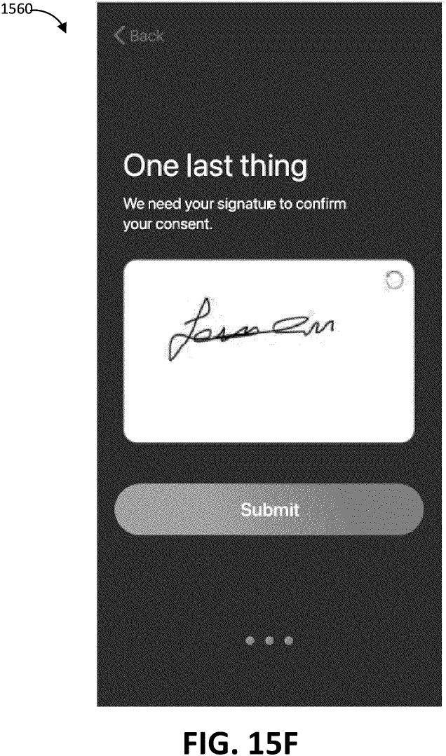

FIG. 15A illustrates an embodiment of an example user interface 1510 for enrolling a potential participant in a clinical trial. In some embodiments, the user interface 1510 is presented via an app that is running on the potential participant's device, such as, for example, a smart phone, laptop, desktop, or tablet. However, it is recognized that a variety of devices may be utilized to run the app. In other embodiments, the user interface 1510 is presented via a website portal via a browser window or via a device not associated with the potential participant, such as, for example, a clinician device, a clinical trial system device, and so forth. The example embodiment includes a user interface feature that allows for the scanning of a setup or enrollment code. In some embodiments, the setup code may have been provided to the potential participant via an email, SMS message, mailed letter, or other form. The potential participant may utilize a camera feature of the potential participant's device, such as, for example, a smart phone to scan the setup code. The example user interface 1510 also include a user interface feature to allow the potential participant to provide a clinical trial identifier and an email or to access a login user interface if the potential participant already has an account.

FIG. 15B illustrates an embodiment of an example user interface 1520 for collecting enrollment information related to the potential participant. In some embodiments, the information may be manually entered by the potential participant via the potential participant's device and/or automatically populated via backend lookups in a database system, such as for example, via a lookup associated with the setup code. The example user interface 1520 may also include a timing element feature that provides an estimate of the amount of time left before the enrollment is complete. In some embodiments, the amount of time may be indicated via an alphanumeric text or provided in a graphical widget such as a status bar, an hourglass, or a set of progression objects illustrating the different phases of the enrollment process as well as an indicator on which of the different phases have been completed. In the example user interface 1520, additional user interface elements are provided such as, for example, an element that allows the potential participant to initiate a question or help session where the potential participant may ask a question via a user interface portal such as, for example, a chat window or a message window. In addition, the user interface 1520 may provide contact information such as phone numbers that can be contacted for questions. The example user interface 1520 may also include an information feature that allows a potential participant to obtain additional information about the clinical trial, the app, or the overall process. For example, the information may allow the potential participant to see which default language has been selected and then change to a different language. The information may also provide contact information, study information, as well as any required information related to the clinical trial. The example user interface 1520 may also include an interface feature that allows the potential participant to save and exit where the information already provided by the potential participant is saved and the potential participant may continue at a later time, whether on the same device or via another device. The example user interface 1520 may also include a navigation feature that allows the potential participant to move to previous or future portions of the process. In some embodiments, the navigation is restricted by rules that require that certain portions of the process be completed before the potential participant is allowed to move to the next portion of the process or require that certain portions of the process that have been completed cannot be undone or modified once submitted. It is recognized that the navigation feature may include alphanumeric and/or graphical text and may be linked to progression objects that indicate which stage of the process the potential participant is currently in. It is recognized that one or all of the user interface features may be provided on multiple user interface screens such as, for example, the help feature, the information feature, the navigation feature, the progression objects, the timing element feature, and/or the save feature.

FIG. 15C illustrates an embodiment of an example user interface 1530 for presenting electronic information associated with the specific clinical trial period. The user interface 1530 may include information regarding potential risks associated with the clinical trial, such as, for example, side effects and/or required regulatory information related to the clinical trial. In addition, the information provided may include links to other sources such as, for example, electronic dictionaries, translators, and other resources that provide information about the information presented in the example user interface 1530. In some embodiments, the information provided in the user interface 1530 may be stored on the back-end system and associated with the particular clinical trial that corresponds to the setup code and/or information provided by the potential participant. For example, certain information may be presented based on the information provided by the potential participant whereas that same information may not be provided to other potential participants based on their respective provided information.

FIG. 15D illustrates an embodiment of an example user interface 1540 for managing medical data stored by the clinical trial risk platform 100 and associated with the clinical trial. For example, some clinical trials may require the storage of actual medical samples, whereas other clinical trials may require the storage of data related to a potential participants medical information. The example user interface 1540 electronically provides required information regarding various options related to the storage of such medical samples or medical data as well as user interface features for the potential participant to select how such medical data or medical samples are stored by the clinical trial risk platform 100.

FIG. 15E illustrates an embodiment of an example user interface 1550 for providing an electronic signature, a hand-drawn signature, or an uploaded signature that confirms one or more items in the enrollment process. For example, the example user interface 1550 may include a user interface feature for allowing the potential participant to indicate that the potential participant has reviewed the information in the form, has had the opportunity to ask questions, or has provided required or preferred consents or confirmations. The clinical trial risk platform 100 may include stored rules on which consents and confirmations may be required for the potential participant based on information related to the potential participant as well as which consents and confirmations are preferred but not required.

FIG. 15F illustrates an embodiment of an example user interface 1560 for obtaining a signature from the potential participant. It is recognized that the signature feature may be associated with a third-party electronic signature service, such as, for example, DocuSign or HelloSign. In some embodiments, the example user interface 1560 may also include authentication or verification features that review the signature information provided by the potential participant to determine whether the signature will be accepted by the clinical trial risk platform 100.

### Actions

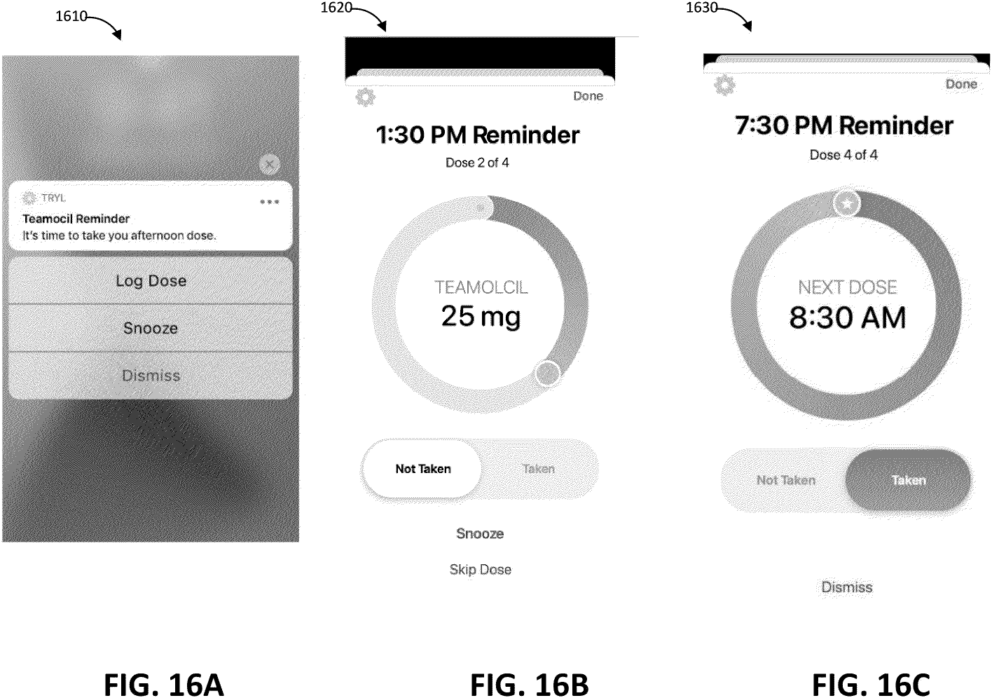

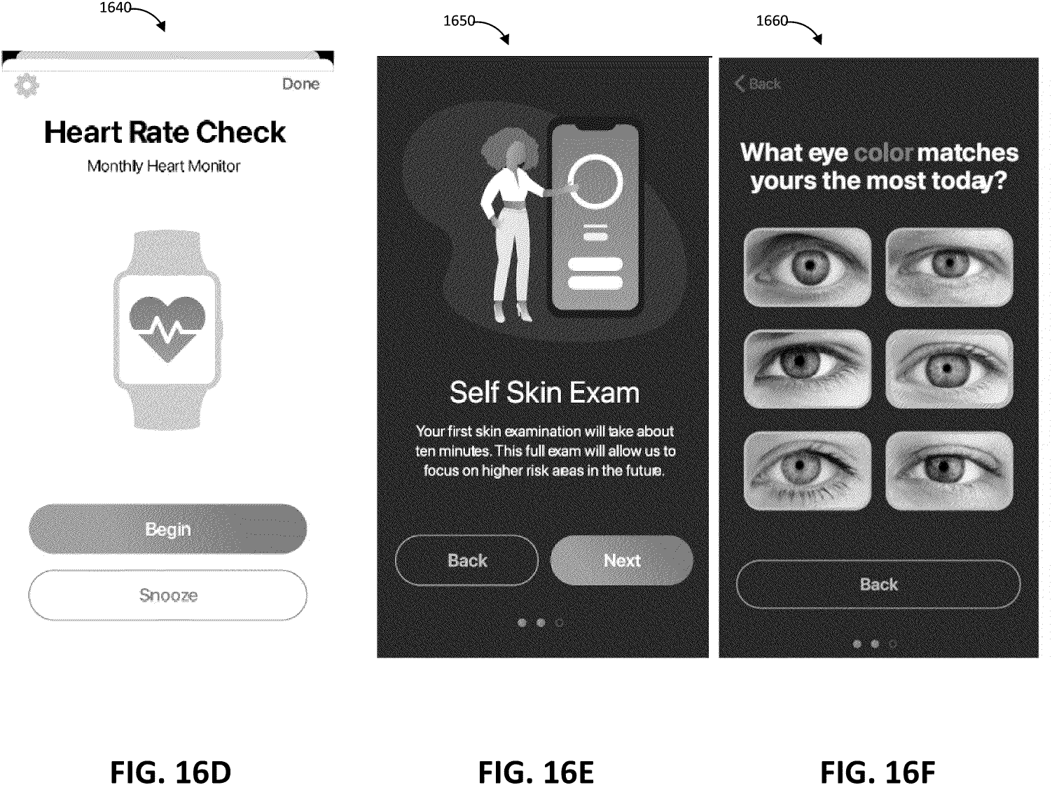

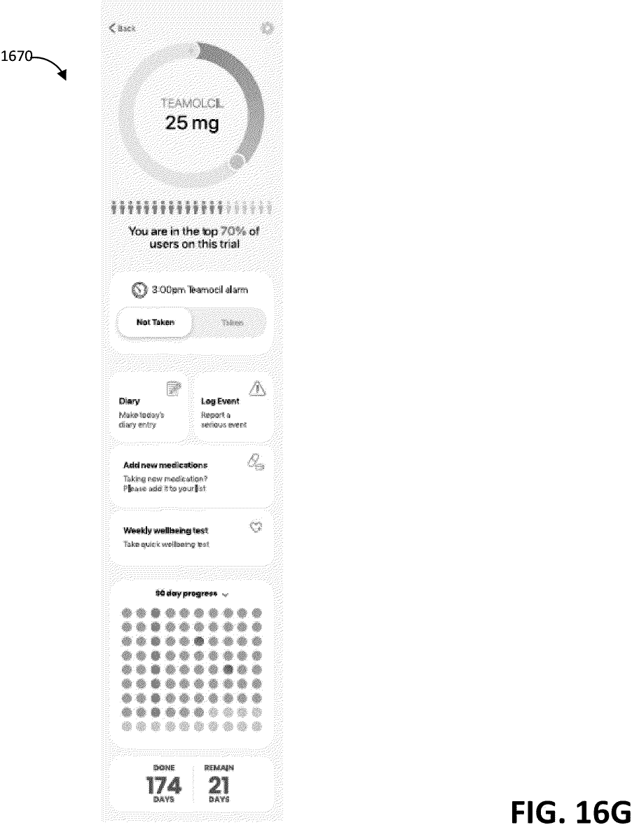

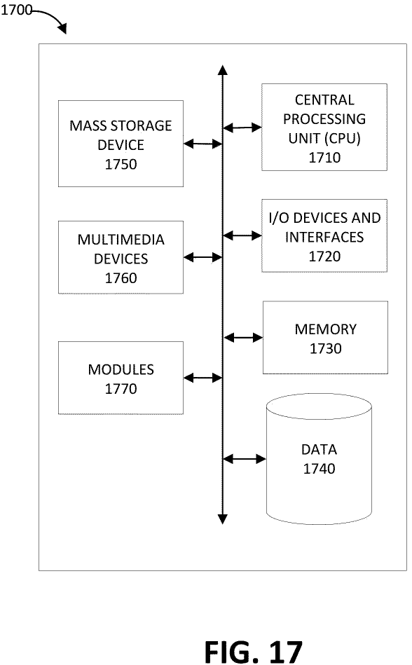

FIG. 16A illustrates an embodiment of an example user interface 1610 for tracking various actions and events required or preferred by the clinical trial protocol based on rules stored on the clinical trial risk platform 100. For example, the example user interface 1610 may include, for example, reminders to take particular medication or to perform certain activities, such, as, for example, to perform certain exercises or take certain measurements. The example user interface 1620 includes user interface elements that allow a participant to log the taking of a dose of a certain medication, to snooze the reminder such that the reminder will be provided at a later time again to the participant, or to dismiss the reminder. In some embodiments, the rules may not allow for the snoozing or dismissing of a reminder, whereas in other embodiments, the rules may trigger certain interventions if certain actions are not completed within an indicated timeframe. In some embodiments, users can use fast logging or "one click" to interact with events from a user interface. The events may include, for example, reminders, interventions, and so forth.

FIG. 16B illustrates an embodiment of an example user interface 1620 for providing a reminder dose for a particular time frame which in the example is a reminder for 1:30 PM to take dose 2 of 4 of a particular medication in a certain amount. The example user interface 1620 includes a unit interface element that allows the participant to indicate via the app whether or not the dose was taken or not taken. While a slider bar is shown, it is recognized that a variety of user interface elements may be used. The example user interface 1620 also includes the snooze feature as well as a skip dose feature that allows the participant to indicate that the participant is skipping the dose

FIG. 16C illustrates an embodiment of an example user interface 1630 for providing a second reminder tied to a different time frame which in the example is a reminder for 7:30 PM to take dose 4 of 4 of the particular medication in a certain amount. The example user interface 1630 indicates that the participant has marked the dose as taken via the slider bar, and the interface displays information on when the next dose is to be taken, which in the example is at 8:30 AM the next morning. The example user interface 1630 also includes a dismiss feature that allows the participant to dismiss the reminder as well as a done feature that allows the participant to indicate that the task has been completed and that the user interface can be exited or closed.

FIG. 16D illustrates an embodiment of an example user interface 1640 for providing a user interface element that allows the participant to perform an activity, which in the example is checking the participant's heart rate. The example user interface 1640 includes a begin element that when activated executes and initiates a heart rate check service that tracks and calculates a heart rate of the participant such as, for example, via the device on which the app is running or via communication with another device that has a sensor that allows for the heart rate to be detected and calculated. The example user interface 1640 also includes a snooze feature that allows the participant to receive a reminder at a later time regarding the same activity.

FIG. 16E illustrates an embodiment of an example user interface 1650 for providing a user interface element that allows the participant to perform a different activity, which in the example is performing a self-examination of the participant's skin. The example user interface 1650 includes instructions on how to perform the skin self-exam, and may also include an activation element that initiates execution of a camera or video function of the device or a device that is in communication with the device running the app and then provides the photo or video to a services that analyzes the photo or video to detect certain features of the skin. In some embodiments, the user interface 1650 may include a feature that initiates a live video conferencing session such that a third party, such as a clinician, may review the participant's skin during the video conference.

FIG. 16F illustrates an embodiment of an example user interface 1660 for providing a user interface element that allows the participant to perform another activity which in the example is determining the eye color of the participant. The example user interface 1670 includes example eye color options for the user such that the participant can choose the graphic that best matches the participants eye color at that time period. The example user interface 1660 may include an activation element that initiates execution of a camera or video function of the device or a device that is in communication with the device running app to take a picture or video of the participant's eye and then provides the photo or video to a service that analyzes the photo or video to determine the eye color. In some embodiments, the user interface 1660 may include a feature that initiates a live video conferencing session such that a third party, such as a clinician, may review the participant's eye color during the video conference.

FIG. 16G illustrates an embodiment of an example user interface 1670 for providing the participant with information about the various activities which in the example is the taking of the required medication. In the example user interface 1670 information is provided that conveys where the participant ranks as to other participants of the same trial and provides information on reminders for particular doses. The example user interface 1670 also includes additional user interface features including a diary feature, a log event feature, an add new medications feature, as well as a weekly well-being test feature. The example user interface 1670 further includes an indication of the status of the participant over a timeframe, such as, over a period of 90 days and may also provide other timing indicators, such as, the number of days completed in the trial and the remaining days in the trial.

### Example System Implementation and Architecture

FIG. 17 is an embodiment of a block diagram showing an example computing system 1700 with example components that could be used to implement one or more of the systems discussed herein including, without limitation, the clinical trial risk platform 100, the API gateway 105, the app/web services system, the authentication/permissioning system 115, the protocol system 120, the medical data system 125, the risk assessment/intervention system 130, the engagement system 135, the machine learning model development system 140, the machine learning model deployment system 145, the participant devices 160, the clinical trial systems 170, the clinician devices 175, the medical center systems 180, and the other third-party service systems 185.

The computing system 1700 may include, for example, one or more personal computers that is IBM, Macintosh, or Linux/Unix compatible or a server or workstation. In some embodiments, the computing system 1700 comprises a server, a laptop computer, a smart phone, a personal digital assistant, a tablet, or a desktop system. In one embodiment, the computing system 1700 includes one or more central processing units (CPUs) 1710 or processors, which may each include a conventional or proprietary microprocessor. The computing system 1700 further includes one or more memories 1730, such as random-access memory (RAM) for temporary storage of information, one or more read only memory (ROM) for permanent storage of information, and one or more mass storage devices 1750, such as a hard drive, diskette, solid state drive, or optical media storage device. Typically, the components of the computing system 1700 are connected to the computer using a standard-based bus system. In different embodiments, the standard-based bus system could be implemented in Peripheral Component Interconnect (PCI), Microchannel, Small Computer System Interface (SCSI), Industrial Standard Architecture (ISA) and Extended ISA (EISA) architectures, for example.

The computing system 1700 may include modules 1770 which may be configured for execution by the CPU 1710 to perform any or all of the processes discussed herein. Depending on the embodiment, certain processes or groups of processes discussed herein may be performed by multiple devices, such as multiple computing systems similar to computing system 1700. The computing system 1700 may also store data 1740 that is accessible by other components of the computing system 1700 or systems external to the computing system 1700. It is recognized that the functionality provided for in the components and modules of computing system 1700 may be combined into fewer components and modules or further separated into additional components and modules.

The computing system 1700 is generally controlled and coordinated by operating system software, such as Windows XP, Windows Vista, Windows 7, Windows 8, Windows 10, Windows 11, Windows Server, Unix, Linux, SunOS, Solaris, Blackberry OS, or other compatible operating systems. In Macintosh systems, the operating system may be any available operating system, such as iOS or MAC OS X. In other embodiments, the computing system 1700 may be controlled by a proprietary operating system. Conventional operating systems control and schedule computer processes for execution, perform memory management, provide file system management, networking, I/O services, and user interfaces, such as a graphical user interface (GUI), among other things.

The example computing system 1700 may include one or more commonly available input/output (I/O) devices and interfaces 1720, such as a keyboard, mouse, touchpad, touchscreen, and printer. In one embodiment, the I/O devices and interfaces 1720 include one or more display devices, such as a monitor, which allows the visual presentation of data to a user. More particularly, a display device provides for the presentation of user interface elements, application software data, reports, benchmarking data, metrics, and/or multimedia presentations, for example. The computing system 1700 may also include one or more multimedia devices 1760, such as speakers, video cards, graphics accelerators, and microphones, for example.

In the embodiment of FIG. 17, the I/O devices and interfaces 1720 provide a communication interface to various external devices. The computing system 1700 may be directly or indirectly electronically coupled to one or more networks, which comprise one or more of a local area network (LAN), wide area network (WAN), and/or the Internet, for example, via one or more wired, wireless, or combination of wired and wireless, communication links. The networks communicate with various computing devices, other electronic devices and systems via wired or wireless communication links, such as the data sources.

In some embodiments, information may be provided to the computing system 1700 over a network from one or more data sources. The data sources may include one or more internal and/or external data sources that provide data. In some embodiments, one or more of the databases or data sources may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Cache), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

In general, the word "module," as used herein, refers to logic embodied in hardware or firmware, or to a collection of software instructions, possibly having entry and exit points, written in a programming language, such as, for example, Java, Lua, C, C#, or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the computing system 1700, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules but may be represented in hardware or firmware. Generally, the modules described herein refer to logical modules that may be combined with other modules or divided into submodules despite their physical organization or storage.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, "can," "could," "might," or "may," unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms "comprising," "including," "having," and the like are synonymous and are used inclusively, in an open- ended fashion, and do not exclude additional elements, features, acts, operations, and so forth. In addition, the term "or" is used in its inclusive sense (and not in its exclusive sense) so that when used, for example, to connect a list of elements, the term "or" means one, some, or all of the elements in the list. In addition, the articles "a," "an," and "the" as used in this application and the appended claims are to be construed to mean "one or more" or "at least one" unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

As used herein, the terms "determine" or "determining" encompass a wide variety of actions. For example, "determining" may include calculating, computing, processing, deriving, generating, obtaining, looking up (for example, looking up in a table, a database, or another data structure), ascertaining and the like via a hardware element without user intervention. Also, "determining" may include receiving (for example, receiving information), accessing (for example, accessing data in a memory) and the like via a hardware element without user intervention. Also, "determining" may include resolving, selecting, choosing, establishing, and the like via a hardware element without user intervention.

As used herein, the terms "provide" or "providing" encompass a wide variety of actions. For example, "providing" may include storing a value in a location of a storage device for subsequent retrieval, transmitting a value directly to the recipient via at least one wired or wireless communication medium, transmitting or storing a reference to a value, and the like. "Providing" may also include encoding, decoding, encrypting, decrypting, validating, verifying, and the like via a hardware element.

As used herein, the terms "message" and "data package" encompasses a wide variety of formats for communicating (for example, transmitting or receiving) information. A message or data package may include a machine-readable aggregation of information such as an XML document, fixed field message, comma separated message, or the like. A message or data package may, in some implementations, include a signal utilized to transmit one or more representations of the information. While recited in the singular, it will be understood that a message or data package may be composed, transmitted, stored, received, and so forth in multiple parts.

As used herein "receive" or "receiving" may include specific algorithms for obtaining information. For example, receiving may include transmitting a request message for the information. The request message may be transmitted via a network as described above. The request message may be transmitted according to one or more well-defined, machine-readable standards which are known in the art. The request message may be stateful in which case the requesting device and the device to which the request was transmitted maintain a state between requests. The request message may be a stateless request in which case the state information for the request is contained within the messages exchanged between the requesting device and the device serving the request. One example of such state information includes a unique token that can be generated by either the requesting or serving device and included in messages exchanged. For example, the response message may include the state information to indicate what request message caused the serving device to transmit the response message.

As used herein "generate" or "generating" may include specific algorithms for creating information based on or using other input information. Generating may include retrieving the input information such as from memory or as provided input parameters to the hardware performing the generating. Once obtained, the generating may include combining the input information. The combination may be performed through specific circuitry configured to provide an output indicating the result of the generating. The combination may be dynamically performed such as through dynamic selection of execution paths based on, for example, the input information, device operational characteristics (for example, hardware resources available, power level, power source, memory levels, network connectivity, bandwidth, and the like). Generating may also include storing the generated information in a memory location. The memory location may be identified as part of the request message that initiates the generating. In some implementations, the generating may return location information identifying where the generated information can be accessed. The location information may include a memory location, network locate, file system location, or the like.

As used herein, "activate" or "activating" may refer to causing or triggering a mechanical, electronic, or electro-mechanical state change to a device. Activation of a device may cause the device, or a feature associated therewith, to change from a first state to a second state. In some implementations, activation may include changing a characteristic from a first state to a second state such as, for example, changing the viewing state of a lens of stereoscopic viewing glasses. Activating may include generating a control message indicating the desired state change and providing the control message to the device to cause the device to change state.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As it is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated. While the embodiments provide various features, examples, screen displays, user interface features, and analyses, it is recognized that other embodiments may be used.

In addition, it is intended that some of the tools, such as those described herein, may be commercially offered under various trademarks that may appear in the text set forth herein. It should be understood that any trademarks included are used to identify the source of certain goods and services, including, for example, the clinical trial risk platform 100. Accordingly, the use of the any trademarks in the figures or text of this application is not, and should not be interpreted as, a generic descriptor of the goods and services described herein.

1. A system for deployment of one or more risk models for determining intervention risk, the system comprising:
one or more processors;
a network communications interface;
a memory; and
computer code stored in the memory, wherein the computer code, when retrieved from the memory and executed by the one or more processors causes the one or more processors to:
access a set of electronic patient medical data associated with a patient and an intervention protocol;
access a set of intervention protocol parameters associated with a medical treatment plan in which the patient is actively engaged;
automatically generate a linked patient data set based on the set of electronic patient medical data and the set of intervention protocol parameters;
apply a deployed machine learning model to generate one or more intervention risk scores associated with a potential harm associated with the patient, wherein the deployed machine learning model is trained using anonymized historical treatment data, wherein the anonymized historical treatment data includes one or more of: historical patient medical data, historical treatment results data; historical patient engagement data; historical clinical data; and
for the one or more intervention risk scores that meet a predetermined threshold indicated in the set of intervention protocol parameters, automatically assess the one or more intervention risk scores to select and initiate an intervention action that reduces the potential harm associated with the patient.

2. The system of claim 1, further comprising computer code stored in the memory, wherein the computer code, when retrieved from the memory and executed by the one or more processors causes the one or more processors to store computer-executable instructions that:
initiate an electronic service that actuates a sensor system to collect medical data associated with the patient.

3. The system of claim 2, wherein the collected medical data includes one more of: blood pressure readings, heart rate, glucose level, or oxygen saturation.

4. The system of claim 1, wherein the set of electronic patient medical data associated with the patient includes one or more of: biometric data collected from the patient, patient response data provided by the patient, periodic reporting data indicating an intake of a medication by the patient as compared to a predetermined dosage.

5. The system of claim 1, wherein the deployed machine learning model employs at least one of: a neural network, an adversarial classifier, a k-nearest neighbor algorithm, linear regression, Support Vector Machines, or a Naive Bayes classifier.

6. The system of claim 1, further comprising computer code stored in the memory, wherein the computer code, when retrieved from the memory and executed by the one or more processors causes the one or more processors to store computer-executable instructions that:
initiate an electronic service that actuates a notification system to send a secured alert in real-time to a medical clinician indicating that assistance is needed for the patient.

7. The system of claim 1, further comprising computer code stored in the memory, wherein the computer code, when retrieved from the memory and executed by the one or more processors causes the one or more processors to store computer-executable instructions that:
initiate an electronic service that actuates a notification system to send a secured alert in real-time to a medical system indicating that the patient is at risk for disengaging with required protocols associated with the medical treatment plan.

8. A computer-implemented method of deploying one or more risk models for determining intervention risk, the computer-implemented method comprising, as implemented by one or more computing devices configured with specific executable instructions to at least:
access a set of electronic patient medical data associated with a patient and an intervention protocol;
access a set of intervention protocol parameters associated with a medical treatment plan in which the patient is actively engaged;
automatically generate a linked patient data set based on the set of electronic patient medical data and the set of intervention protocol parameters;
apply a deployed machine learning model to generate one or more intervention risk scores associated with a potential harm associated with the patient, wherein the deployed machine learning model is trained using anonymized historical treatment data, wherein the anonymized historical treatment data includes one or more of: historical patient medical data, historical treatment results data; historical patient engagement data; historical clinical data; and
for the one or more intervention risk scores that meet a predetermined threshold indicated in the set of intervention protocol parameters, automatically assess the one or more intervention risk scores to select and initiate an intervention action that reduces the potential harm associated with the patient.

9. The computer-implemented method of claim 8 further comprising specific executable instructions that:
initiate an electronic service that actuates a sensor system to collect medical data associated with the patient.

10. The computer-implemented method of claim 9, wherein the collected medical data includes one more of: blood pressure readings, heart rate, glucose level, or oxygen saturation.

11. The computer-implemented method of claim 8, wherein the set of electronic patient medical data associated with the patient includes one or more of: biometric data collected from the patient, patient response data provided by the patient, periodic reporting data indicating an intake of a medication by the patient as compared to a predetermined dosage.

12. The computer-implemented method of claim 8, wherein the deployed machine learning model employs at least one of: a neural network, an adversarial classifier, a k-nearest neighbor algorithm, linear regression, Support Vector Machines, or a Naive Bayes classifier.

13. The computer-implemented method of claim 8 further comprising specific executable instructions that:
initiate an electronic service that actuates a notification system to send a secured alert in real-time to a medical clinician indicating that assistance is needed for the patient.

14. The computer-implemented method of claim 8 further comprising specific executable instructions that:
initiate an electronic service that actuates a notification system to send a secured alert in real-time to a medical system indicating that the patient is at risk for disengaging with required protocols associated with the medical treatment plan.

15. A non-transitory computer storage medium storing computer-executable instructions that, when executed by one or more processors, cause the one or more processors to at least:
access a set of electronic patient medical data associated with a patient and a n intervention protocol;
access a set of intervention protocol parameters associated with a medical treatment plan in which the patient is actively engaged;
automatically generate a linked patient data set based on the set of electronic patient medical data and the set of intervention protocol parameters;
apply a deployed machine learning model to generate one or more intervention risk scores associated with a potential harm associated with the patient, wherein the deployed machine learning model is trained using anonymized historical treatment data, wherein the anonymized historical treatment data includes one or more of: historical patient medical data, historical treatment results data; historical patient engagement data; historical clinical data; and
for the one or more intervention risk scores that meet a predetermined threshold indicated in the set of intervention protocol parameters, automatically assess the one or more intervention risk scores to select and initiate an intervention action that reduces the potential harm associated with the patient.

16. The non-transitory computer storage medium of claim 15, further storing computer-executable instructions that:
initiate an electronic service that actuates a sensor system to collect medical data associated with the patient.

17. The non-transitory computer storage medium of claim 16, wherein the collected medical data includes one more of: blood pressure readings, heart rate, glucose level, or oxygen saturation.

18. The non-transitory computer storage medium of claim 15, wherein the set of electronic patient medical data associated with the patient includes one or more of: biometric data collected from the patient, patient response data provided by the patient, periodic reporting data indicating an intake of a medication by the patient as compared to a predetermined dosage.

19. The non-transitory computer storage medium of claim 15, wherein the deployed machine learning model employs at least one of: a neural network, an adversarial classifier, a k-nearest neighbor algorithm, linear regression, Support Vector Machines, or a Naive Bayes classifier.

20. The non-transitory computer storage medium of claim 15, further storing computer-executable instructions that:
initiate an electronic service that actuates a notification system to send a secured alert in real-time to a medical clinician indicating that assistance is needed for the patient.
